(19) United States
(12) Patent Application Publication
(10) Pub. No.: US 2026/0232496 A1
(43) Pub. Date: Aug. 13, 2026

(54) SHAPED CO-FORM ABSORBENT CORES AND METHODS OF MAKING THE SAME

(71) Applicant: The Procter & Gamble Company, Cincinnati, OH (US)

(72) Inventors: Astrid Annette Sheehan, Symmes Township, OH (US); Lothar Volker Kuntze, Swisttal (DE)

(21) Appl. No.: 19/532,049

(22) Filed: Feb. 6, 2026

Related U.S. Application Data
(60) Provisional application No. 63/757,386, filed on Feb. 12, 2025.

(51) Int. Cl. A61F 13/15; A61F 13/53

(52) U.S. Cl. CPC A61F 13/1565; A61F 13/53; A61F 2013/530233; A61F 2013/530489

(57) ABSTRACT

A method of forming an absorbent core is presented. The method includes advancing a first substrate layer in a machine direction, the first substrate layer forming a first side of the absorbent core. The method further includes feeding a first stream of absorbent material comprising superabsorbent particles and thermoplastic filaments toward the first substrate layer and depositing the first stream of absorbent material onto the first substrate layer through at least one discharge nozzle, wherein the at least one discharge nozzle is configured to rotate about an axis orthogonal to the machine direction, and wherein the deposited first stream of absorbent material forms a first absorbent material layer. The method also includes applying a second substrate layer to form a second side of the absorbent core opposite the first side of the absorbent core.

### CROSS REFERENCE TO RELATED APPLICATION

This application claims the benefit, under 35 U.S.C. §119(e), of U.S. Provisional Application No. 63/757,386, filed February 12, 2025, the entire disclosure of which is incorporated herein by reference.

### FIELD

### The present disclosure is generally directed to shaped co-form absorbent cores useful in personal care absorbent articles. The present disclosure is also directed to methods of making shaped co-form absorbent cores.

### BACKGROUND

Personal care absorbent articles are primarily designed to absorb and retain body exudates such as urine, fecal material, blood, and menses, with additional desired attributes including low leakage of the exudates from the absorbent article and a dry feel to the wearer of the absorbent article. By preventing leakage of the exudates from the absorbent article, the absorbent article may prevent the body exudates from contaminating a wearer’s or caregiver’s clothing or other articles, such as bedding, that can come in contact with the wearer.

Absorbent cores typically designed to take-up and store liquid within absorbent articles. Many absorbent cores contain multiple absorbent materials such as superabsorbent material and pulp fluff or other fibrous absorbent material. Each type of absorbent material helps to impart a range of properties useful in absorbing and retaining liquid bodily exudates. For example, pulp fluff or other fibrous absorbent material may absorb liquid more quickly than superabsorbent material, and the superabsorbent material may be able retain more liquid per particle than pulp fluff. Many advances have been made to absorbent cores, and particularly to the superabsorbent material of absorbent cores. Some current absorbent cores may now have absorbent material comprising mostly superabsorbent material and further comprising only a small portion of other absorbent material (co-form cores). Other current absorbent cores comprise only superabsorbent material as the absorbent material. Absorbent cores which contain mostly or only superabsorbent material are generally thinner, better able to move with a wearer, and generally more comfortable as compared to other types of absorbent cores. To improve fit and comfort, such absorbent cores may also be shaped – having a crotch region that is narrower than the front and back – in order to fit well between the legs of the wearer and remain relatively narrow even when wet. Methods of making such absorbent cores, however, typically require removing crotch portions, resulting in waste of absorbent material and substrate material. Thus, further improvements to such shaped co-form absorbent cores are a continued area of exploration to increase their performance.

### SUMMARY

Aspects of the present disclosure may solve one or more of the problems discussed above by providing methods of forming an absorbent core. The present disclosure provides, in part, a method comprising advancing a first substrate layer in a machine direction, the first substrate layer forming a first side of the absorbent core. The method further comprises feeding a first stream of absorbent material comprising superabsorbent particles and thermoplastic filaments toward the first substrate layer and depositing the first stream of absorbent material onto the first substrate layer through at least one discharge nozzle, wherein the at least one discharge nozzle is configured to rotate about an axis orthogonal to the machine direction, and wherein the deposited first stream of absorbent material forms a first absorbent material layer. The method also comprises applying a second substrate layer to form a second side of the absorbent core opposite the first side of the absorbent core.

The present disclosure provides, in part, a method of forming an absorbent core, the method comprising advancing a first substrate layer in a machine direction, the first substrate layer forming a first side of the absorbent core and feeding a first stream of absorbent material comprising superabsorbent particles and thermoplastic filaments toward the first substrate layer. The method further comprises depositing the first stream of absorbent material onto the first substrate layer through at least one discharge nozzle, wherein the at least one discharge nozzle comprises an applicator or a plurality of applicators, wherein the applicator or the plurality of applicators move about an axis orthogonal to the machine direction, and wherein the deposited first stream of absorbent material forms a first absorbent material layer, and applying a second substrate layer to form a second side of the absorbent core opposite the first side of the absorbent core.

The present disclosure provides, in part, a method of forming an absorbent core, the method comprising advancing a first substrate layer in a machine direction, the first substrate layer forming a first side of the absorbent core, feeding a first stream of absorbent material comprising superabsorbent particles and thermoplastic filaments toward the first substrate layer, depositing the first stream of absorbent material onto the first substrate layer through at least one discharge nozzle, wherein the at least one discharge nozzle comprises a plurality of applicators, wherein the plurality of applicators move relative to each other in a by-pass manner, and wherein the deposited first stream of absorbent material forms a first absorbent material layer, and finally applying a second substrate layer to form a second side of the absorbent core opposite the first side of the absorbent core.

### BRIEF DESCRIPTION OF THE DRAWINGS

The above-mentioned and other features and advantages of the present disclosure, and the manner of attaining them, will become more apparent and the disclosure itself will be better understood by reference to the following description of example forms of the disclosure taken in conjunction with the accompanying drawings, wherein:

FIG. 1 is a plan view of an example absorbent article in the form of a taped diaper, garment-facing surface facing the viewer, in a flat laid-out state;

FIG. 2 is a plan view of the example absorbent article of FIG. 1, wearer-facing surface facing the viewer, in a flat laid-out state;

FIG. 3 is a front perspective view of the absorbent article of FIGS. 1 and 2 in a fastened position;

FIG. 4 is a front perspective view of an absorbent article in the form of a pant;

FIG. 5 is a rear perspective view of the absorbent article of FIG. 4;

FIG. 6 is a plan view of the absorbent article of FIG. 4, laid flat, with a garment-facing surface facing the viewer;

FIG. 7 is a cross-sectional view of the absorbent article taken about line 7—7 of FIG. 6;

FIG. 8 is a cross-sectional view of the absorbent article taken about line 8—8 of FIG. 6;

FIG. 9 is a plan view of an example absorbent core or an absorbent article;

FIG. 10 is a cross-sectional view, taken about line 10—10, of the absorbent core of FIG. 9;

FIG. 11 is a cross-sectional view, taken about line 11—11, of the absorbent core of FIG. 9;

FIG. 12 is a plan view of an example absorbent article of the present disclosure that is a sanitary napkin;

FIG. 13 is a plan view of an example absorbent core having a shaped absorbent material deposition area;

FIG. 14 is a cross-sectional view of an example absorbent core with an internal structuring web material;

FIG. 15 is a cross-sectional view of a further example absorbent core with an internal structuring web material;

FIG. 16 is an exploded cross-sectional view of an example absorbent article having a masking layer;

FIG. 17 is a schematic illustration of a method of making an absorbent article of the present disclosure;

FIG. 18 is a schematic illustration of chute having a discharge nozzle;

FIGS. 19-21 are plan views of the progression of chute and/or discharge nozzle depositing absorbent material onto a first substrate layer;

FIGS. 22 and 23 are plan views of first substrate layers showing discharge nozzles with a plurality of movable applicators;

FIG. 24 is a schematic illustration of a further method of making an absorbent article of the present disclosure;

FIG. 25 is plan view of a first substrate layer showing a discharge nozzle with a channel forming applicator; and

FIG. 26 is a cross-sectional view of a package of absorbent articles.

### DETAILED DESCRIPTION

Various non-limiting forms of the present disclosure will now be described to provide an overall understanding of the principles of the structure, function, manufacture, and use of the shaped co-form cores and methods of making the same disclosed herein. One or more examples of these non-limiting forms are illustrated in the accompanying drawings. Those of ordinary skill in the art will understand that the shaped co-form cores and methods of making the same described herein and illustrated in the accompanying drawings are non-limiting example forms and that the scope of the various non-limiting forms of the present disclosure are defined solely by the claims. The features illustrated or described in connection with one non-limiting form may be combined with the features of other non-limiting forms. Such modifications and variations are intended to be included within the scope of the present disclosure.

### General Description of an Absorbent Article

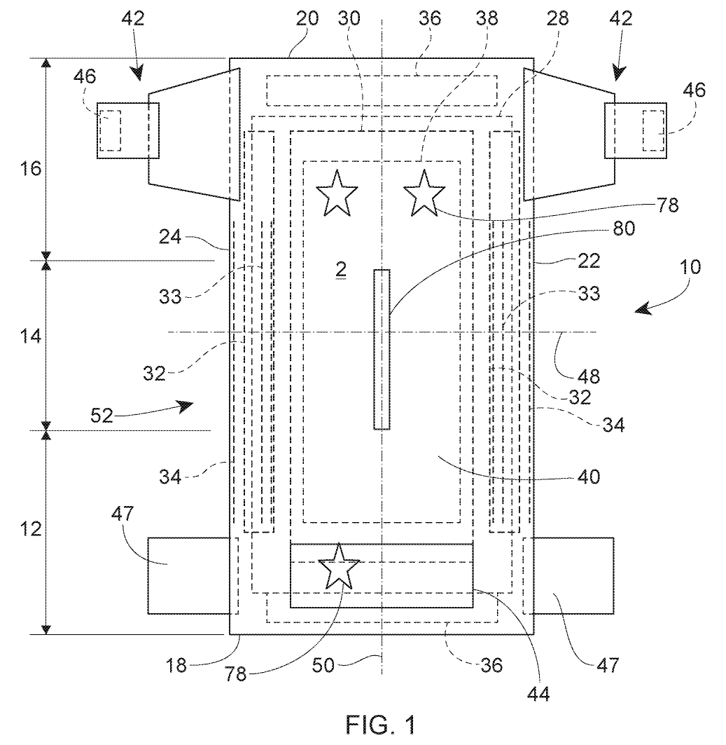

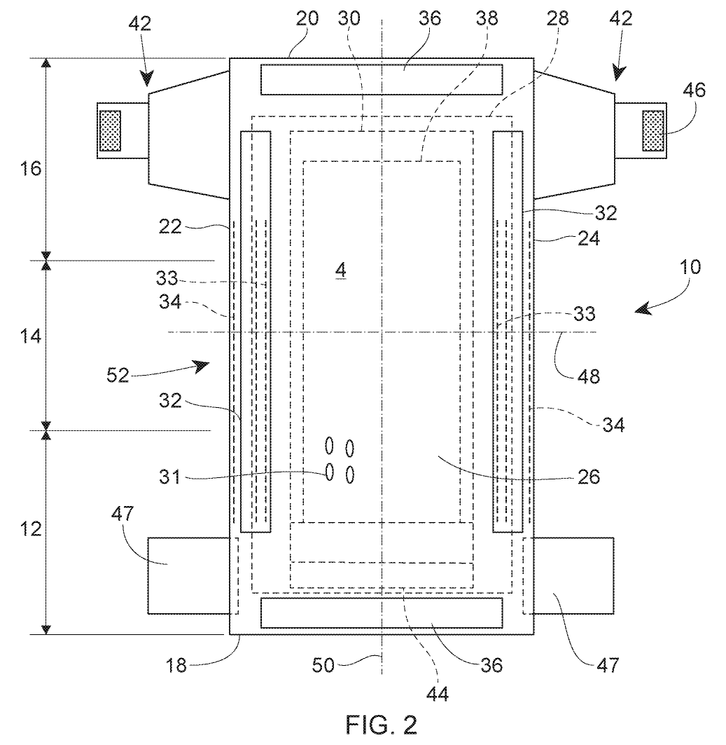

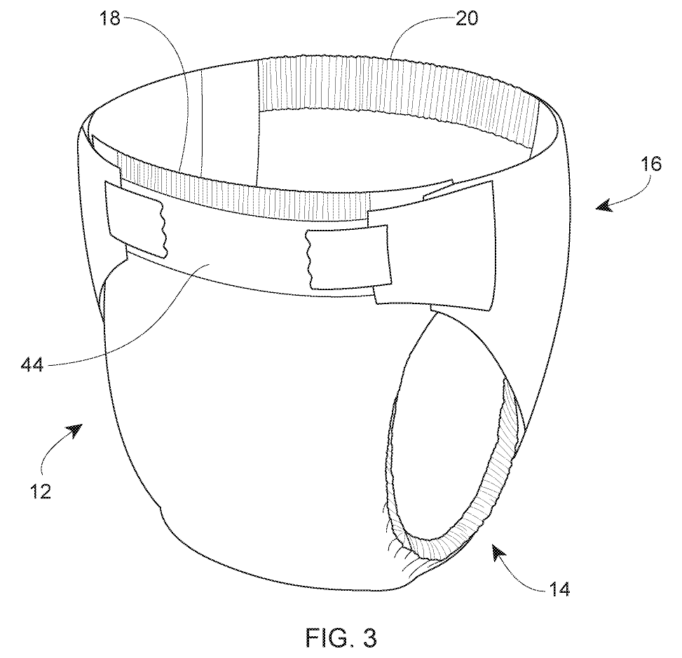

An example absorbent article 10 according to the present disclosure, shown in the form of a taped diaper, is represented in FIGS. 1-3. FIG. 1 is a plan view of the example absorbent article 10, garment-facing surface 2 facing the viewer in a flat, laid-out state (i.e., no elastic contraction). FIG. 2 is a plan view of the example absorbent article 10 of FIG. 1, wearer-facing surface 4 facing the viewer in a flat, laid-out state. FIG. 3 is a front perspective view of the absorbent article 10 of FIGS. 1 and 2 in a fastened configuration. The absorbent article 10 of FIGS. 1-3 is shown for illustration purposes only as the present disclosure may be used for making a wide variety of diapers, including adult incontinence products, pants, or other absorbent articles, such as sanitary napkins and absorbent pads, for example.

The absorbent article 10 may comprise a front waist region 12, a crotch region 14, and a back waist region 16. The crotch region 14 may extend intermediate the front waist region 12 and the back waist region 16. The front wait region 12, the crotch region 14, and the back waist region 16 may each be 1/3 of the length of the absorbent article 10. The absorbent article 10 may comprise a front end edge 18, a back end edge 20 opposite to the front end edge 18, and longitudinally extending, transversely opposed side edges 22 and 24 defined by the chassis 52.

The absorbent article 10 may comprise a liquid permeable topsheet 26, a liquid impermeable backsheet 28, and an absorbent core 30 positioned at least partially intermediate the topsheet 26 and the backsheet 28. The absorbent article 10 may also comprise one or more pairs of barrier leg cuffs 32 with or without elastics 33, one or more pairs of leg elastics 34, one or more elastic waistbands 36, and/or one or more acquisition materials 38. The acquisition material or materials 38 may be positioned intermediate the topsheet 26 and the absorbent core 30. An outer cover material 40, such as a nonwoven material, may cover a garment-facing side of the backsheet 28. The absorbent article 10 may comprise back ears 42 in the back waist region 16. The back ears 42 may comprise fasteners 46 and may extend from the back waist region 16 of the absorbent article 10 and attach (using the fasteners 46) to the landing zone area or landing zone material 44 on a garment-facing portion of the front waist region 12 of the absorbent article 10. The absorbent article 10 may also have front ears 47 in the front waist region 12. The absorbent article 10 may have a central lateral (or transverse) axis 48 and a central longitudinal axis 50. The central lateral axis 48 extends perpendicular to the central longitudinal axis 50. When discussing methods of manufacturing absorbent articles herein, the longitudinal direction parallel with the central longitudinal axis 50 may be referred to as the machine direction, and the lateral direction parallel with the central lateral axis 48 may be referred to as the cross-machine direction.

In other instances, the absorbent article may be in the form of a pant having permanent or refastenable side seams. Suitable refastenable seams are disclosed in U.S. Pat. Appl. Pub. No. 2014/0005020 and U.S. Pat. No. 9,421,137.

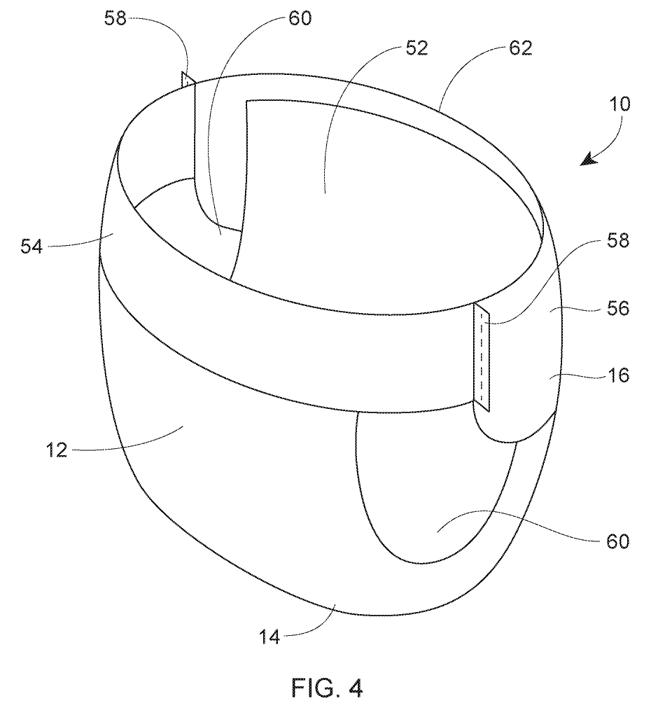

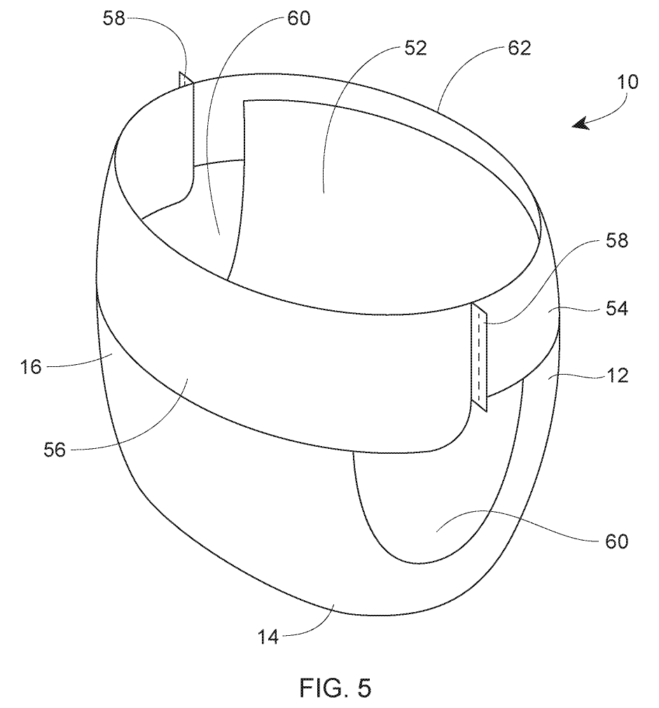

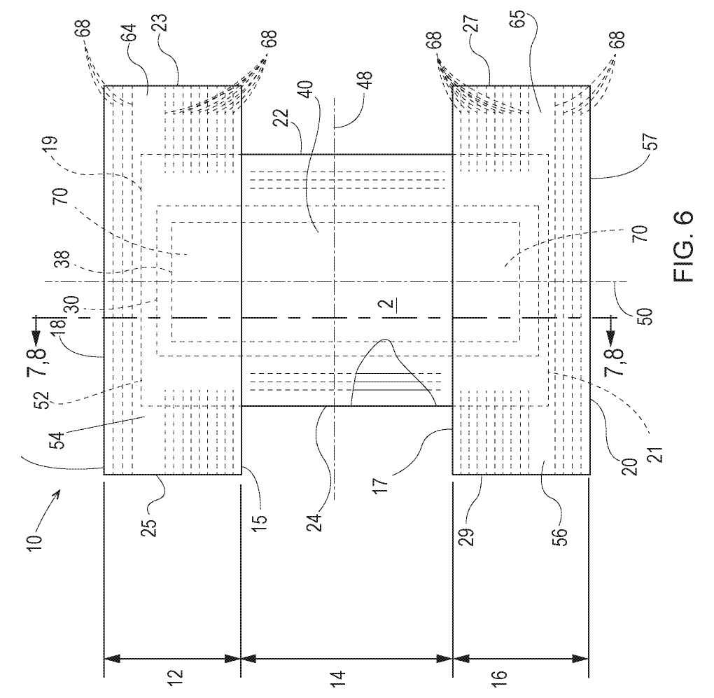

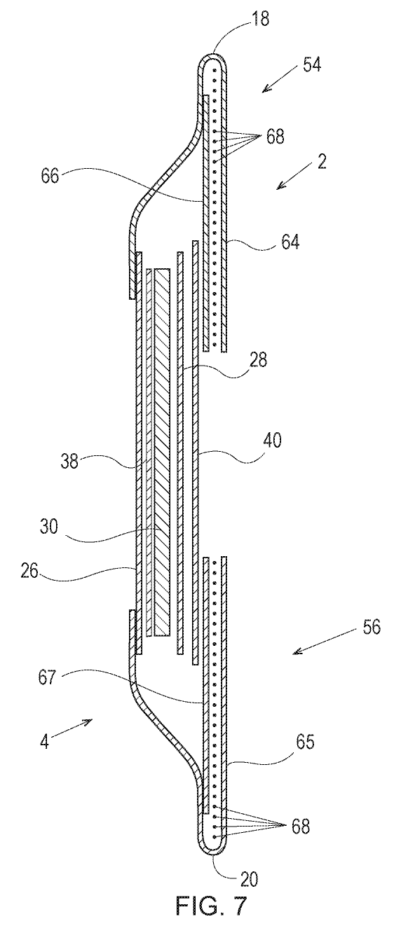

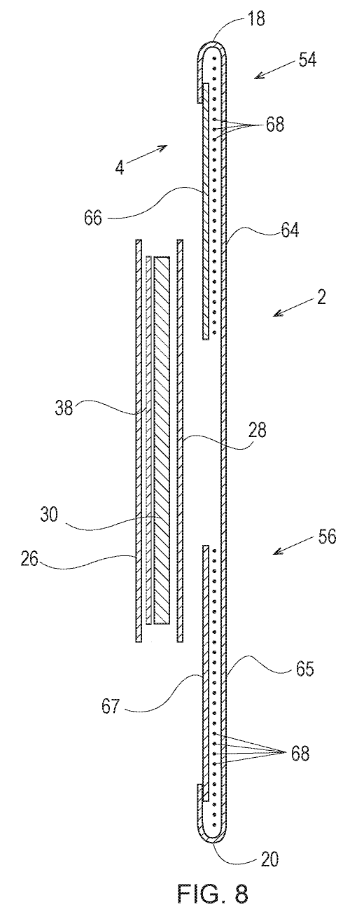

Referring to FIGS. 4-8, an example absorbent article 10 in the form of a pant is illustrated. FIG. 4 is a front perspective view of the absorbent article 10. FIG. 5 is a rear perspective view of the absorbent article 10. FIG. 6 is a plan view of the absorbent article 10, laid flat, with the garment-facing surface facing the viewer. Elements of FIG. 4-8 having the same reference number as described above with respect to FIGS. 1-3 may be the same element (e.g., absorbent core 30). FIG. 7 is an example cross-sectional view of the absorbent article taken about line 7—7 of FIG. 6. FIG. 8 is an example cross-sectional view of the absorbent article taken about line 8—8 of FIG. 6. FIGS. 7 and 8 illustrate example forms of front and back belts 54, 56. The absorbent article 10 may have a front waist region 12, a crotch region 14, and a back waist region 16. Each of the regions 12, 14, and 16 may be 1/3 of the length of the absorbent article 10. The absorbent article 10 may have a chassis 52 (sometimes referred to as a central chassis or central panel) comprising a topsheet 26, a backsheet 28, and an absorbent core 30 disposed at least partially intermediate the topsheet 26 and the backsheet 28, and an optional acquisition material 38, similar to that as described above with respect to FIGS. 1-3. The absorbent article 10 may comprise a front belt 54 in the front waist region 12 and a back belt 56 in the back waist region 16. The chassis 52 may be joined to a wearer-facing surface 4 of the front and back belts 54, 56 or to a garment-facing surface 2 of the belts 54, 56. Side edges 23 and 25 of the front belt 54 may be joined to side edges 27 and 29, respectively, of the back belt 56 to form two side seams 58. The side seams 58 may be any suitable seams known to those of skill in the art, such as butt seams or overlap seams, for example. When the side seams 58 are permanently formed or refastenably closed, the absorbent article 10 in the form of a pant has two leg openings 60 and a waist opening circumference 62. The side seams 58 may be permanently joined using adhesives or bonds, for example, or may be refastenably closed using hook and loop fasteners, for example.

### Belts

Referring to FIGS. 7 and 8, the front and back belts 54 and 56 may comprise front and back inner belt layers 66 and 67 and front and back outer belt layers 64 and 65 having an elastomeric material (e.g., strands 68 or a film (which may be apertured)) disposed at least partially therebetween. The elastic elements 68 or the film may be relaxed (including being cut) to reduce elastic strain over the absorbent core 30 or, may alternatively, run continuously across the absorbent core 30. The elastics elements 68 may have uniform or variable spacing therebetween in any portion of the belts. The elastic elements 68 may also be pre-strained the same amount or different amounts. The front and/or back belts 54 and 56 may have one or more elastic element free zones 70 where the chassis 52 overlaps the belts 54, 56. In other instances, at least some of the elastic elements 68 may extend continuously across the chassis 52.

The front and back inner belt layers 66, 67 and the front and back outer belt layers 64, 65 may be joined using adhesives, heat bonds, pressure bonds or thermoplastic bonds. Various suitable belt layer configurations can be found in U.S. Pat. No. 9,072,632.

Front and back belt end edges 55 and 57 may extend longitudinally beyond the front and back chassis end edges 19 and 21 (as shown in FIG. 6) or they may be co-terminus. The front and back belt side edges 23, 25, 27, and 29 may extend laterally beyond the chassis side edges 22 and 24. The front and back belts 54 and 56 may be continuous (i.e., having at least one layer that is continuous) from belt side edge to belt side edge (e.g., the transverse distances from 23 to 25 and from 27 to 29). Alternatively, the front and back belts 54 and 56 may be discontinuous from belt side edge to belt side edge (e.g., the transverse distances from 23 to 25 and 27 to 29), such that they are discrete.

As disclosed in U.S. Pat. No. 7,901,393, the longitudinal length (along the central longitudinal axis 50) of the back belt 56 may be greater than the longitudinal length of the front belt 54, and this may be particularly useful for increased buttocks coverage when the back belt 56 has a greater longitudinal length versus the front belt 54 adjacent to or immediately adjacent to the side seams 58.

The front outer belt layer 64 and the back outer belt layer 65 may be separated from each other, such that the layers are discrete or, alternatively, these layers may be continuous, such that a layer runs continuously from the front belt end edge 55 to the back belt end edge 57. This may also be true for the front and back inner belt layers 66 and 67 – that is, they may also be longitudinally discrete or continuous. Further, the front and back outer belt layers 64 and 65 may be longitudinally continuous while the front and back inner belt layers 66 and 67 are longitudinally discrete, such that a gap is formed between them – a gap between the front and back inner and outer belt layers 64, 65, 66, and 67 is shown in FIG. 7 and a gap between the front and back inner belt layers 66 and 67 is shown in FIG. 8. In some configurations shown in FIG. 7 and 8, portions of the outer belt layers 64, 65 may be folded over onto the inner belt layers, respectively. In addition, as shown in FIG. 7, portions of the outer belt layers 64, 65 may also be folded over onto the chassis 52.

The front and back belts 54 and 56 may include slits, holes, and/or perforations providing increased breathability, softness, and a garment-like texture. Underwear-like appearance can be enhanced by substantially aligning the waist and leg edges at the side seams 58 (see FIGS. 4 and 5).

The front and back belts 54 and 56 may comprise graphics (see e.g., 78 of FIG. 1). The graphics may extend substantially around the entire circumference of the absorbent article 10 and may be disposed across side seams 58 and/or across proximal front and back belt edges 15 and 17; or, alternatively, adjacent to the seams 58 and/or proximal front and back belt edges 15 and 17 in the manner described in U.S. Pat. No. 9,498, 389 to create a more underwear-like article. The graphics may also be discontinuous.

Alternatively, instead of attaching belts 54 and 56 to the chassis 52 to form a pant, discrete side panels may be attached to side edges of the chassis 22 and 24. Suitable forms of pants comprising discrete side panels are disclosed in U.S. Pat. Nos. 6,645,190; 8,747,379; 8,372,052; 8,361,048; 6,761,711; 6,817,994; 8,007,485; 7,862,550; 6,969,377; 7,497,851; 6,849,067; 6,893,426; 6,953,452; 6,840,928; 8,579,876; 7,682,349; 7,156,833; and 7,201,744.

### Topsheet

The topsheet 26 is the part of the absorbent article 10 that is in contact with the wearer’s skin. The topsheet 26 may be joined to portions of the backsheet 28, the absorbent core 30, the barrier leg cuffs 32, and/or any other layers as is known to those of ordinary skill in the art. The topsheet 26 may be compliant, soft-feeling, and non-irritating to the wearer's skin. Further, at least a portion of, or all of, the topsheet may be liquid permeable, permitting liquid bodily exudates to readily penetrate through its thickness. A suitable topsheet may be manufactured from a wide range of materials, such as porous foams, reticulated foams, apertured plastic films, woven materials, nonwoven materials, woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers. The topsheet may have one or more layers. Where the topsheet comprises more than one layer of material, the layers may have the same dimensions, or the layers may have different dimensions. One layer of a multi-layer topsheet may have a length and/or a width that is different than another layer of the multi-layered topsheet. The topsheet may be apertured (FIG. 2, element 31), may have any suitable three-dimensional features, and/or may have a plurality of embossments (e.g., a bond pattern). The topsheet may comprise a variable basis weight nonwoven material, such as those described in U.S. Pat. Appl. No. 2017/0191198. The topsheet may be apertured by overbonding a material and then rupturing the overbonds through ring rolling, such as disclosed in U.S. Patent No. 5,628,097, to Benson et al., issued on May 13, 1997, and disclosed in U.S. Pat. Appl. Publication No. US 2016/0136014 to Arora et al. Any portion of the topsheet may be coated with a skin care composition, an antibacterial agent, a surfactant, and/or other beneficial agents. The topsheet may be hydrophilic or hydrophobic or may have hydrophilic and/or hydrophobic portions or layers. If the topsheet is hydrophobic, typically apertures will be present so that bodily exudates may pass through the topsheet. The topsheet may comprise a one or more layer hydroentangled material with or without apertures. The topsheet may comprise a variable basis weight nonwoven material.

### Backsheet

The backsheet 28 is generally that portion of the absorbent article 10 positioned proximate to the garment-facing surface of the absorbent core 30. The backsheet 28 may be joined to portions of the topsheet 26, the outer cover material 40, the absorbent core 30, and/or any other layers of the absorbent article by any attachment methods known to those of skill in the art. The backsheet 28 prevents, or at least inhibits, the bodily exudates absorbed and contained in the absorbent core 10 from soiling articles such as bedsheets, undergarments, and/or clothing. The backsheet is typically liquid impermeable, or at least substantially liquid impermeable. The backsheet may, for example, be or comprise a thin plastic film, such as a thermoplastic film having a thickness of about 0.012 mm to about 0.051 mm. Other suitable backsheet materials may include breathable materials, such as films, which permit vapors to escape from the absorbent article, while still preventing, or at least inhibiting, bodily exudates from passing through the backsheet. The backsheet 28 may be coterminous with the outer cover material 40.

### Outer Cover Material

The outer cover material (sometimes referred to as a backsheet nonwoven) 40 may comprise one or more nonwoven materials joined to the backsheet 28 and that covers the backsheet 28. The outer cover material 40 forms at least a portion of the garment-facing surface 2 of the absorbent article 10 and effectively “covers” the backsheet 28 so that film is not present on the garment-facing surface 2. The outer cover material 40 may comprise a bond pattern, apertures, and/or three-dimensional features. The outer cover material 40 may be a hydroentangled nonwoven material or a variable basis weight nonwoven material. Such a material may have one or more layers.

### Barrier Leg Cuffs/Leg Elastics

Referring to FIGS. 1 and 2, for example, the absorbent article 10 may comprise one or more pairs of barrier leg cuffs 32 and one or more pairs of leg elastics 34. The barrier leg cuffs 32 may be positioned laterally inboard of leg elastics 34. Each barrier leg cuff 32 may be formed by a piece of material which is bonded to the absorbent article 10 so it can extend upwards from a wearer-facing surface 4 of the absorbent article 10 and provide improved containment of body exudates approximately at the junction of the torso and legs of the wearer. The barrier leg cuffs 32 are delimited by a proximal edge joined directly or indirectly to the topsheet and/or the backsheet and a free terminal edge, which is intended to contact and form a seal with the wearer’s skin. The barrier leg cuffs 32 may extend at least partially between the front end edge 18 and the back end edge 20 of the absorbent article 10 on opposite sides of the central longitudinal axis 50 and may be at least present in the crotch region 14. The barrier leg cuffs 32 may each comprise one or more elastics 33 (e.g., elastic strands or strips) near or at the free terminal edge. These elastics 33 cause the barrier leg cuffs 32 to help form a seal around the legs and torso of a wearer. The leg elastics 34 extend at least partially between the front end edge 18 and the back end edge 20. The leg elastics 34 essentially cause portions of the absorbent article 10 proximate to the chassis side edges 22, 24 to help form a seal around the legs of the wearer. The leg elastics 34 may extend at least within the crotch region 14. The barrier leg cuffs 32 may comprise one or more layers of nonwoven material. The nonwoven material may be hydroentangled.

### Elastic Waistband

Referring to FIGS. 1 and 2, the absorbent article 10 may comprise one or more elastic waistbands 36. The elastic waistbands 36 may be positioned on the garment-facing surface 2 or the wearer-facing surface 4. Alternatively, the elastic waistbands may be positioned intermediate the topsheet and the backsheet. As an example, a first elastic waistband 36 may be present in the front waist region 12 near the front end edge 18 and a second elastic waistband 36 may be present in the back waist region 16 near the back end edge 20. The first and/or second waistband may be coterminous with the front end edge and/or back end edge, or the waistband(s) may be offset from the respective end edge. The waistband(s) may overlap a front and/or rear edge of the absorbent core. The elastic waistbands 36 may aid in sealing the absorbent article 10 around a waist of a wearer and at least inhibiting bodily exudates from escaping the absorbent article 10 through the waist opening circumference. In some instances, an elastic waistband may fully surround the waist opening circumference of an absorbent article. The elastic waistband may comprise an elastic film joined to the topsheet and a nonwoven material covering the elastic film. In other instances, the elastic waistband may comprise an elastic film sandwiched between two nonwoven materials. The elastic film may be ultrasonically bonded, or otherwise bonded or attached, to the one or more nonwoven materials. The one or more nonwoven materials may be hydroentangled. The elastic film and/or the nonwoven materials may be preactivated (i.e., activated prior to being joined together) or the formed elastic film/nonwoven laminate may be activated post laminate formation.

### Acquisition Materials

Referring to FIGS. 1, 2, 7, and 8, one or more acquisition materials 38 may be present at least partially intermediate the topsheet 26 and the absorbent core 30. The acquisition materials 38 are typically hydrophilic materials that provide significant wicking of bodily exudates. These materials may dewater the topsheet 26 and quickly move bodily exudates into the absorbent core 30. The acquisition materials 38 may comprise one or more nonwoven materials, foams, formed films, apertured films, apertured formed films, cellulosic materials, cross-linked cellulosic materials, air laid cellulosic nonwoven materials, spunlace materials, or combinations thereof, for example. The acquisition material may be rectangular or may be shaped, such as hourglass shaped, oval, or asymmetrical along the longitudinal or lateral axis. Typically, an acquisition material 38 may have a width and length that are smaller than the width and length of the topsheet 26. The acquisition material may be a secondary topsheet in the feminine pad context. The acquisition materials may have one or more channels as described above with reference to the absorbent core 30 (including the embossed version). The channels in the acquisition material may align or not align with channels in the absorbent core 30. In an example, a first acquisition material may comprise a nonwoven material and as second acquisition material may comprise a cross-linked cellulosic material.

### Landing Zone

Referring to FIGS. 1 and 2, the absorbent article 10 may have a landing zone area 44 that is formed in a portion of the garment-facing surface 2 of the outer cover material 40. The landing zone area 44 may be in the back waist region 16 if the absorbent article 10 fastens from front to back or may be in the front waist region 12 if the absorbent article 10 fastens back to front. In some instances, the landing zone 44 may be or may comprise one or more discrete nonwoven materials that are attached to a portion of the outer cover material 40 in the front waist region 12 or the back waist region 16 depending upon whether the absorbent article fastens in the front or the back. In essence, the landing zone 44 is configured to receive the fasteners 46 and may comprise, for example, a plurality of loops configured to be engaged with, a plurality of hooks on the fasteners 46, or vice versa.

### Wetness Indicator/Graphics

Referring to FIG. 1, the absorbent articles 10 of the present disclosure may comprise graphics 78 and/or wetness indicators 80 that are visible from the garment-facing surface 2. The graphics 78 may be printed on the landing zone 40, the backsheet 28, and/or at other locations. The wetness indicators 80 are typically applied to the absorbent core facing side of the backsheet 28, so that they can be contacted by bodily exudates within the absorbent core 30. In some instances, the wetness indicators 80 may form portions of the graphics 78. For example, a wetness indicator may appear or disappear and create/remove a character within some graphics. In other instances, the wetness indicators 80 may coordinate (e.g., same design, same pattern, same color) or not coordinate with the graphics 78. The wetness indicator(s) 80 may at least partially overlap a core channel. Wetness indicator(s) 80 may be disposed between core channels, such that there is no overlap between the wetness indicator(s) 80 and the core channels.

### Front and Back Ears

Referring to FIGS. 1 and 2, as referenced above, the absorbent article 10 may have front and/or back ears 47, 42 in a taped diaper context. Only one set of ears may be required in most taped diapers. The single set of ears may comprise fasteners 46 configured to engage the landing zone or landing zone area 44. If two sets of ears are provided, in most instances, only one set of the ears may have fasteners 46, with the other set being free of fasteners. Any ears with fasteners may be configured to engage an opposing ear or the landing zone or landing zone area 44. One set of ears may comprise primary fasteners and the other set of ears may comprise secondary fasteners. The ears, or portions thereof, may be elastic or may have elastic panels. In an example, an elastic film or elastic strands may be positioned intermediate a first nonwoven material and a second nonwoven material. One or more of the first and second nonwoven materials may be hydroentangled. The elastic film may or may not be apertured. The ears may be shaped. The ears may be integral (e.g., extension of the outer cover material 40, the backsheet 28, and/or the topsheet 26) or may be discrete components attached to a chassis 52 of the absorbent article on a wearer-facing surface 4, on the garment-facing surface 2, or intermediate the two surfaces 4, 2. The back ears may comprise an elastic film sandwiched between two nonwoven materials forming a laminate. The elastic film and/or the nonwoven materials may be pre-activated (i.e., activated prior to being joined together) and the laminate may be joined by ultrasonic bonds. Such laminates are disclosed in U.S. Pat. No. 10,485,713. In other instances, the elastic film may not be preactivated and instead the laminate may be activated after the laminate is formed. Such laminates may also be ultrasonically bonded.

### Masking Layer

One or more masking layers or materials may be provided in the absorbent articles 10. A masking layer may be a layer that provides a cushiony feel when the absorbent article is touched from the garment-facing surface 2 or the wearer-facing surface 4. The masking layer may “mask” a grainy feel potentially caused by the absorbent material 72, such as superabsorbent polymers. The masking layer may “mask” bodily exudates from being visible when viewing the wearer-facing surface 4 or the garment-facing surface 2 of the absorbent article 10. The masking layer may have a basis weight in the range of about 15 gsm to about 50 gsm or about 15 gsm to about 40 gsm. The masking layer may comprise one or more nonwoven materials (e.g., a hydroentangled nonwoven material), foams, pulp layers, and/or other suitable materials. The masking layer may be the outer cover material 40. The masking layer may be the layer forming the garment-facing side or the wearer-facing side of the core bag 74. The masking layer may be a separate material positioned intermediate the garment-facing side of the core bag 74 and the liquid impermeable backsheet 28.

### Absorbent Core

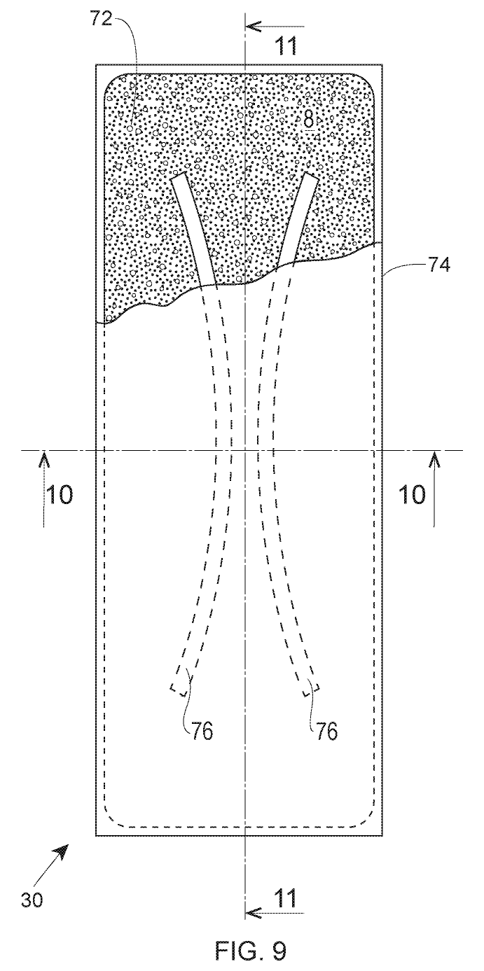

As used herein, the term “absorbent core” refers to a component of the absorbent article 10 configured to absorb and contain liquid such as urine received by the absorbent article. The absorbent core thus typically has a high absorbent capacity. An example absorbent core 30 is schematically shown in FIGS. 9-11. The absorbent core comprises an absorbent material 72. The absorbent core typically comprises a core wrap 74 that encloses or sandwiches the absorbent material 72. As used herein, “absorbent core” does not include any acquisition-distribution systems, topsheet, or backsheet of the absorbent article.

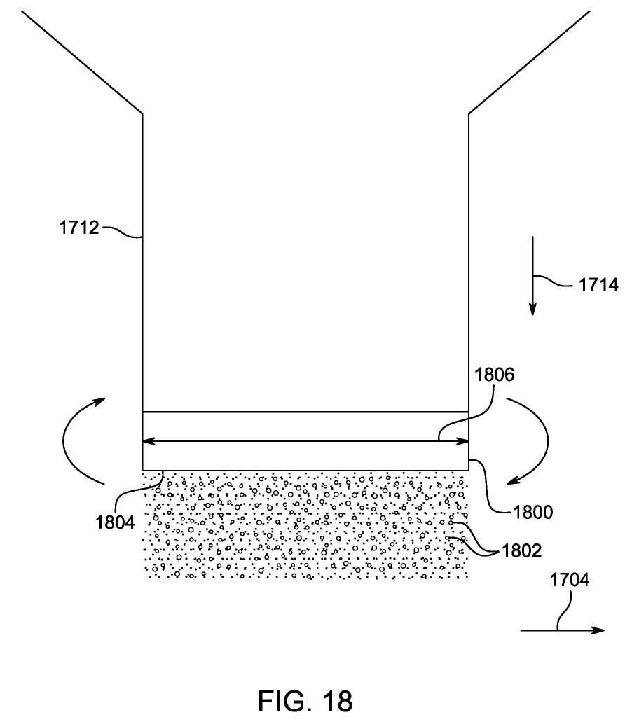

The core wrap 74 may be a single material that is folded and attached to itself, or it may comprise a separate top layer and bottom layer that may be bonded, adhesively joined, or otherwise joined together. The top and bottom layers of the core wrap 74 (also referred to herein as the first substrate material 1702 and the second substrate material 1724) may be the same or different. The core wrap may wrap around the absorbent material 72 in a c-wrap configuration, as shown in FIG. 16. The example absorbent core 30 shown in isolation in FIGS. 9-11 is in the dry state (before use). The absorbent core may typically have a generally rectangular shape as defined by its longitudinal edges and transversal front edge and back edge, or may have other shapes such as trapezoidal, T-shaped, I-shaped, or hourglass shaped. In a form, the longitudinal edges of the absorbent core may form at least portions of the longitudinal side edges 22, 24 of the absorbent article. In another form, the longitudinal edges of the absorbent core may be entirely offset from the longitudinal side edges 22, 24 of the absorbent article.

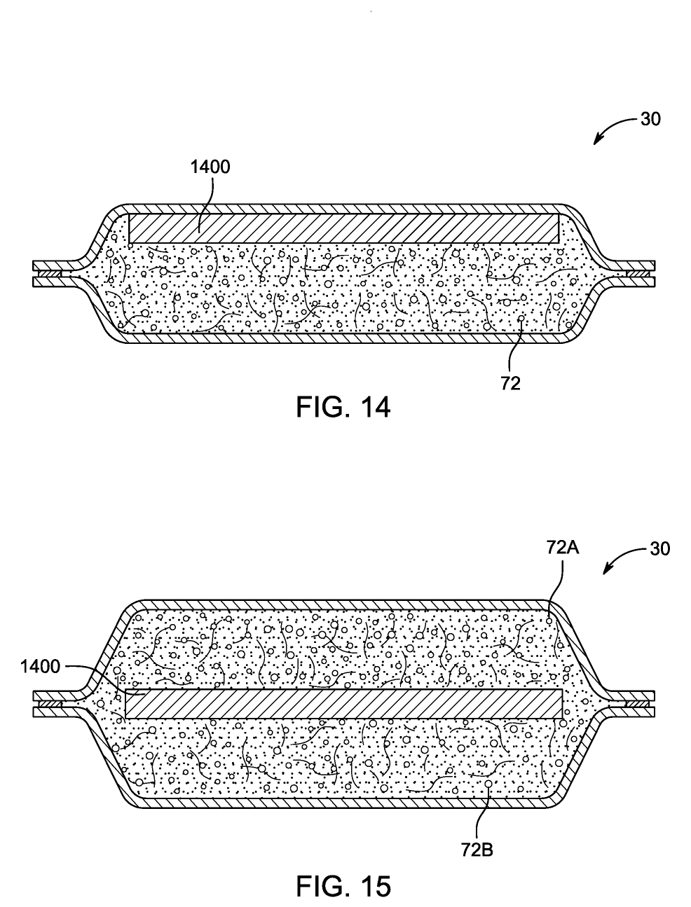

The absorbent core 30 may comprise absorbent material 72 deposited as a single layer of absorbent material 72 or as two or more layers of absorbent material 72. The layer(s) may be deposited to have a generally rectangular outline, as represented in FIG. 9. The absorbent material 72 layer(s) may also have a non-rectangular perimeter (“shaped” core). In particular, the absorbent material 72 may define a tapering along its width towards the central region of the core (or “dog-bone” shape), as shown for example in FIG. 13. In this way, the absorbent material deposition area may have a relatively narrow width in an area of the core intended to be placed in the crotch region of the absorbent article. This may provide, for example, better wearing comfort. Other shapes can also be used such as a “T” or “Y” or “hourglass” for the area of the absorbent material. The one or more layers of absorbent material may comprise virgin materials and/or recycled materials.

The absorbent material 72 typically comprises superabsorbent material 1300 which are optionally mixed with fibrous material 1302. When composed at least partially of superabsorbent materials 1300, such superabsorbent materials can be selected from natural, synthetic, and modified natural polymers and materials. The superabsorbent materials can be inorganic materials, such as silica gels, or organic compounds, such as cross-linked polymers. “Superabsorbent polymer” or “SAP” refers herein to absorbent materials, typically cross-linked polymeric materials, that can absorb at least 10 times their weight of an aqueous 0.9% saline solution as measured using the Centrifuge Retention Capacity (CRC) test (EDANA method WSP 241.2.R3 (12)). The SAP may in particular have a CRC value of at least 20 g/g, in particular of from about 20 g/g to about 40 g/g. “Superabsorbent polymer particles”, as used herein, refers to a superabsorbent polymer material which is in particulate form so as to be flowable in the dry state.

When the absorbent material 72 is composed at least partially of fibrous material 1302, various types of wettable, hydrophilic fibers and/or filaments may be used in the absorbent core 30. Examples of suitable fibrous material include natural fibers, cellulosic fibers, synthetic fibers composed of cellulose or cellulose derivatives, such as rayon fibers; inorganic fibers composed of an inherently wettable material, such as glass fibers; synthetic fibers and/or filaments made from inherently wettable thermoplastic polymers, such as particular polyester or polyamide fibers, or composed of non-wettable thermoplastic polymers, such as polyolefin fibers and/or filaments which have been hydrophilized by suitable means. The fibers and/or filaments may be hydrophilized, for example, by treatment with a surfactant, treatment with silica, treatment with a material which has a suitable hydrophilic moiety and is not readily removed from the fiber, or by sheathing the non-wettable, hydrophobic fiber with a hydrophilic polymer during or after formation of the fiber.

The absorbent material 72 content of the absorbent core 30 may comprise mostly superabsorbent material 1300, by weight of the absorbent material 72 of the absorbent core 30. The absorbent material 72 content of the absorbent core 30 may comprise greater than 65% superabsorbent material, greater than 75% superabsorbent material, greater than 85% superabsorbent material, greater than 90% superabsorbent material, greater than 95% superabsorbent material, by weight of the absorbent material 72 of the absorbent core 30. The remaining absorbent material 72 content may comprise fibrous material 1302, such as cellulosic fibers, or any other suitable absorbent material. The absorbent material 72 content of the absorbent core 30 may comprise 100% superabsorbent material, providing a cellulose-free (air-felt free) absorbent core 30.

Various absorbent core designs comprising high amounts of SAP have been proposed in the past, see for example in U.S. Pat. No. 5,599,335 (Goldman), EP1,447,066 (Busam), WO95/11652 (Tanzer), U.S. Pat. Appl. Pub. No. 2008/0312622A1 (Hundorf), WO2012/052172 (Van Malderen). In particular, the SAP printing technology as disclosed in U.S. Pat. No. 7,838,722 (Blessing), U.S. Pat. No. 9,072,634 and U.S. Pat. No. 8,206,533 (both to Hundorf et al.) may be used. The present disclosure however is not limited to a particular type of absorbent core. The absorbent core may also comprise one or more glues such as an auxiliary glue applied between the internal surface of one (or both) of the core wrap layers and the absorbent material to reduce leakage of SAP outside the core wrap. A micro-fibrous adhesive net may also be used in air-felt free cores as described in the above Hundorf references. These glues are not represented in the Figures for simplicity. Other core constructions comprising a high loft nonwoven substrate, such as a carded nonwoven layer, having a porous structure into which SAP particles have been deposited, may also be used in the present disclosure.

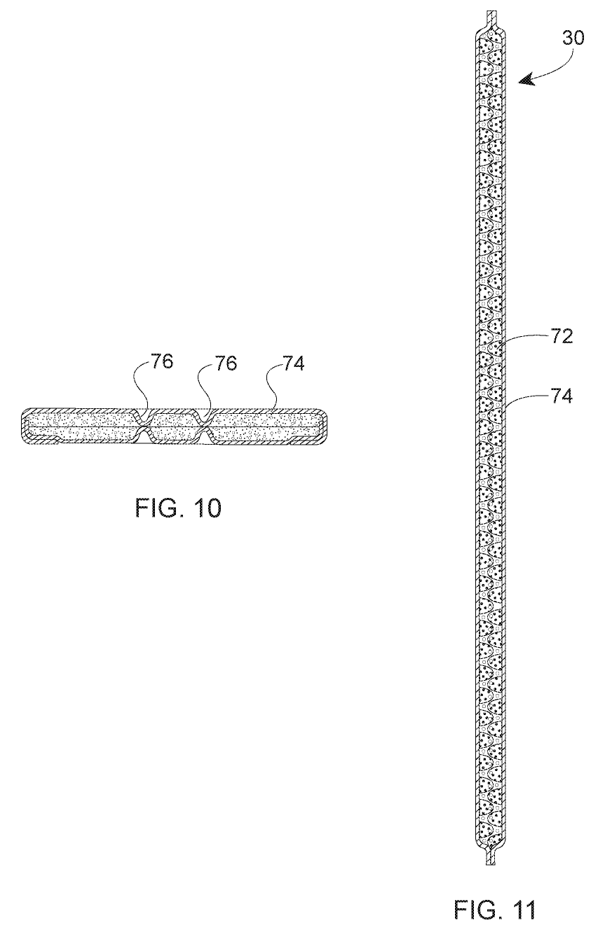

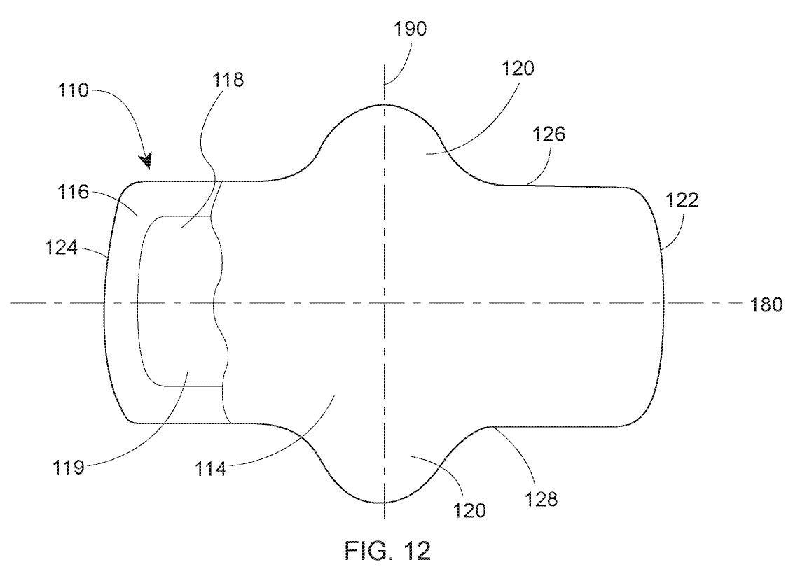

The absorbent material 72 may be deposited as a continuous layer within the core wrap. The absorbent material 72 may also be present discontinuously, for example, as individual pockets or stripes of absorbent material enclosed within the core wrap and separated from each other by absorbent material-free junction areas. A continuous layer of absorbent material 72, in particular of SAP, may also be obtained by combining two absorbent layers having complimentary discontinuous absorbent material application pattern, wherein the resulting layer is substantially continuously distributed across the absorbent material area, as illustrated in FIGS. 10-11. As for example taught in U.S. Pat. Appl. Pub. No. 2008/0312622A1 (Hundorf), each absorbent material layer may thus comprise a pattern having absorbent material land areas and absorbent material-free junction areas, wherein the absorbent material land areas of the first layer correspond substantially to the absorbent material-free junction areas of the second layer and vice versa.

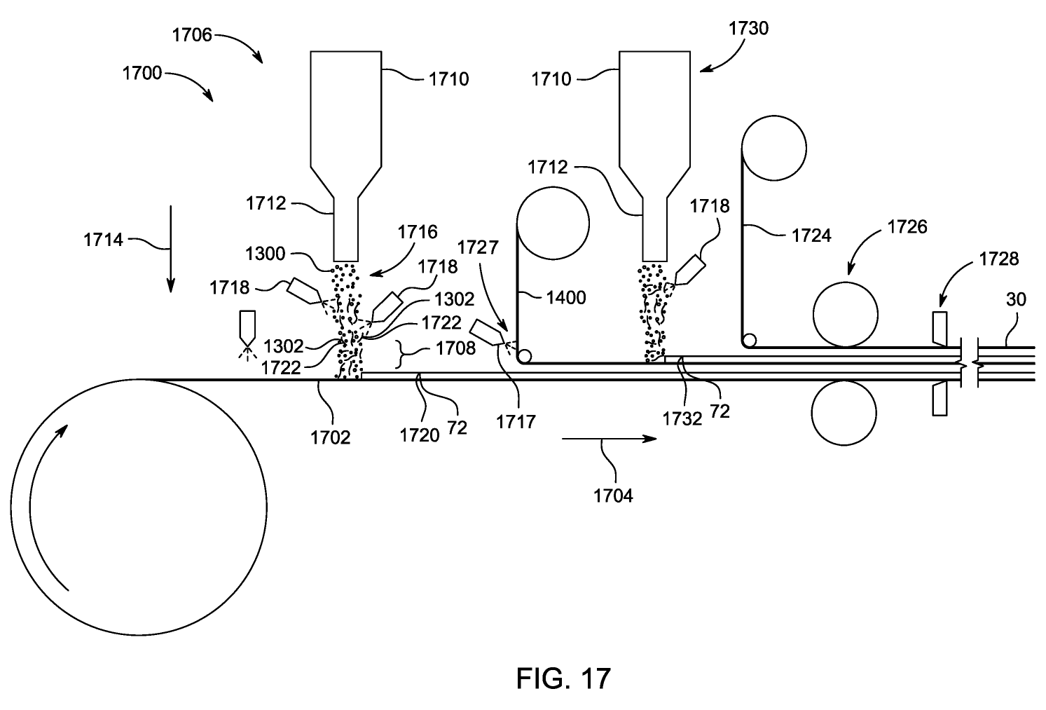

The basis weight (amount deposited per unit of surface) of the absorbent material 72 may also be varied to create a profiled distribution of absorbent material, in particular in the longitudinal direction to provide more absorbency towards the center and the middle of the core, but also in the transversal direction, or both directions of the core. The absorbent core may also comprise one or more longitudinally (or otherwise) extending channels 76, which are areas of the absorbent layer substantially free of absorbent material 72 within the absorbent material layer. The top side of the core wrap may be advantageously bonded to the bottom side of the core by adhesive, mechanical or ultra-sonic bonding through these material-free areas. The one or more channels may be permanent – meaning the bonding of the top side of the core wrap to the bottom side of the core through the channel is resistant to liquid – or temporary – meaning the bonding through the channel is susceptible to releasing in the presence of liquid. Example disclosures of such channels in an airfelt-free core can be found in U.S. Pat. No. 9,789,011. One or more channels may also be formed in absorbent cores comprising a mix of cellulose fibers and SAP particles. These channels may embody any suitable shapes and any suitable number of channels may be provided. In other instances, the absorbent core may be embossed to create the impression of channels, with absorbent material 72 remaining within the channels. The absorbent core in FIGS. 9-11 is merely an example absorbent core. Many other absorbent cores with or without channels are also within the scope of the present disclosure. Where the absorbent core 30 comprises at least two distinctive layers of absorbent material, such as a top layer 72A and a bottom layer 72B, for example as shown in FIG. 15, a channel or channels may be formed in only one layer. For example, a channel may be formed in the top layer 72A, and the top core wrap material may be joined to the internal structuring web material 1400.

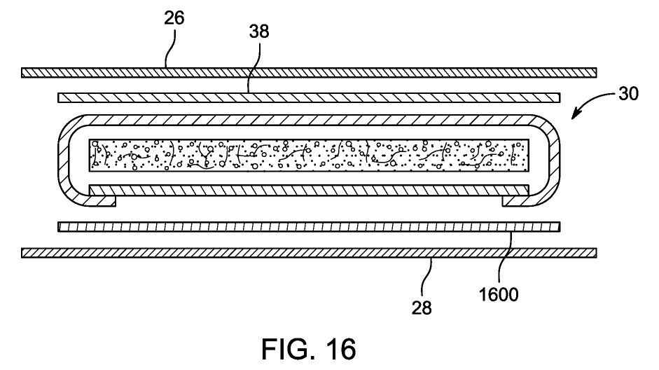

An internal structuring web material 1400 may be disposed within the absorbent core 30 as shown in FIGS. 14 and 15. The internal structuring web material 1400 may be a nonwoven material, and in particular a lofty nonwoven material. The internal structuring web material 1400 may be formed from a spunbond material or a spunbond-meltblown-spunbond (SMS) material, a through-air bonded carded web, a chemically-bonded nonwoven material, a spunlace material, and combinations thereof. The internal structuring web material 1400 may have a basis weight of between about 10 gsm and about 120 gsm.

If a masking layer 1600 is present, as shown in FIG. 16, the absorbent core 30 may be disposed on the masking layer 1600 and superposed over the backsheet 28. The masking layer 1600 may be bonded to the backsheet 28, for example, by adhesive. In some embodiments, a masking layer 1600 may not be present and the absorbent core 30 may directly contact the backsheet 28 and may be directly bonded to the backsheet. However, it is to be understood that the absorbent core 30 may be in contact with, and not bonded with, the backsheet 28 and remain within the scope of this disclosure.

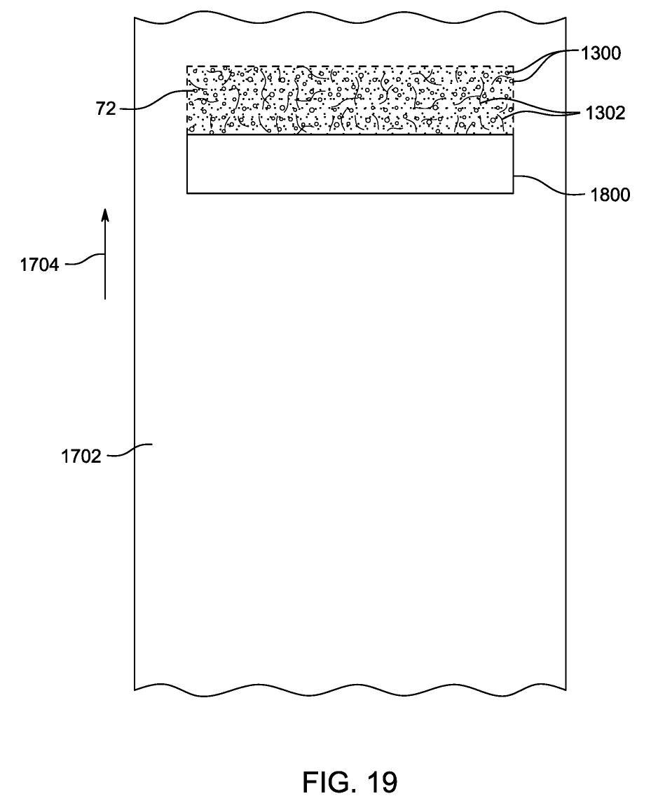

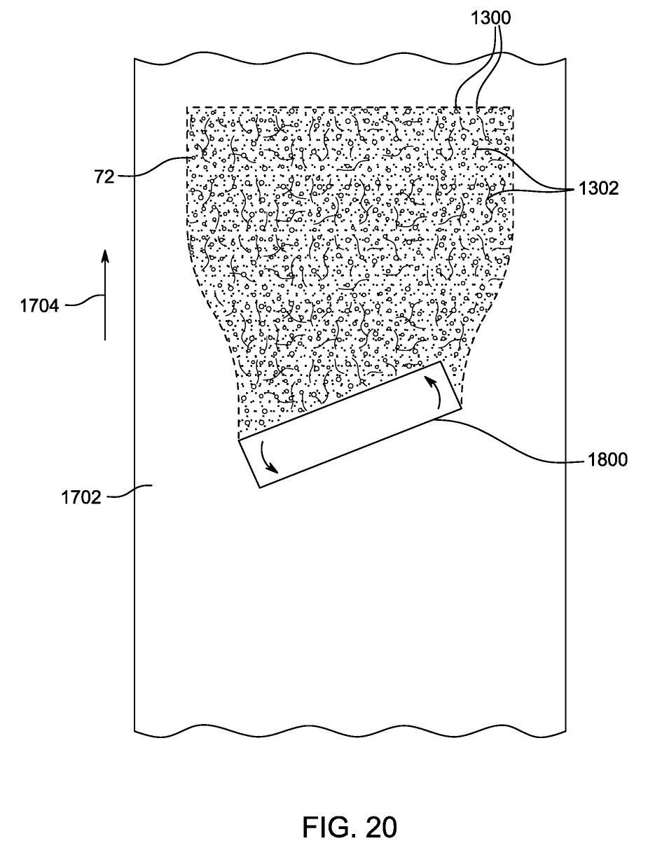

Absorbent cores 30 according to the present disclosure may be formed according to the processes disclosed herein, such as processes 1700, 1800 detailed in FIGS. 17 and 18. Such absorbent cores 30 formed by processes 1700, 1800 may provide greater thinness, flexibility, superabsorbent material capture, intake and/or rewet performance, and less overall material usage than absorbent structures formed by different processes and/or comprising different material or different relative amounts of material.

FIG. 17 is an exemplary schematic depiction of absorbent core formation process 1700 for forming absorbent cores such as absorbent cores 30. Process 1700 includes unwinding first substrate layer 1702 and moving the first substrate layer 1702 in a machine direction 1704. The first substrate layer 1702 may be a core wrap 74 material of an absorbent core 30. The first substrate layer 1702 may form a bottom layer of the core wrap 74, a top layer of the core wrap 74, or may form the entirety of the core warp 74 of the absorbent core 30. The first substrate layer 1702 moves in the machine direction 1704 and arrives at an absorbent material deposition station 1706. At the absorbent material deposition station 1706, absorbent material 72 – such as superabsorbent material 1300 - is intermixed with fibrous material 1302 prior to depositing onto the first substrate layer 1702, for example in a mixing region 1708, and ultimately deposits onto the first substrate layer 1702. The absorbent material 72 may be intermixed with the fibrous material 1302 using air.

The absorbent material 72 flows from hopper 1710 and through chute 1712 toward the first substrate layer 1702. The hopper 1710 may be a bulk solid pump or feeder configured to maintain a consistent flow of the absorbent material 72 through the absorbent material deposition station 1706. The flow rate of the absorbent material 72 out of the hopper 1710 may be adjustable such that the hopper 1710 can deliver different amounts of absorbent material 72, resulting in different basis weights of absorbent material 72 in the finished absorbent cores 30. Such differences in basis weights of absorbent material 72 may allow the formed absorbent cores 30 to be used in different absorbent end uses –such as in diapers, feminine hygiene articles, adult incontinence articles, and the like.

The chute 1712 may comprise a discharge nozzle 1800 (as seen in FIG. 18) that is shown oriented in the vertical direction 1714 such that the absorbent material 72, shown as individual particles 1802 in FIG. 18, exits the discharge nozzle 1800 falling substantially in the vertical direction 1714. The absorbent material 72 may be fed through the absorbent material deposition station 1706 by gravity, without any pneumatic force, or pneumatic force may be used to transport the absorbent material 72. As used herein, the vertical direction 1714 is used to denote a direction perpendicular to the first substrate layer 1702. The machine direction 1704 is a direction parallel within the first substrate layer 1702 and, accordingly, is perpendicular to the vertical direction 1714. In examples where the first substrate layer 1702 is oriented in a horizontal direction with respect to gravity (e.g. perpendicular to the direction of gravity), the vertical direction 1714 may be substantially aligned with respect to gravity. However, in other examples, the vertical direction 1714 may be at an angle with respect to gravity –for example angles of up to 25 degrees difference with respect to gravity may be suitable for the vertical direction 1714. Accordingly, in such embodiments, the first substrate layer 1702 may not be moving in a horizontal direction with respect to gravity and the absorbent material 72 would fall toward the first substrate layer 1300 having a direction including a component in both the vertical direction 1714 and the machine direction 1704 (or potentially opposite the machine direction 1704).

In general, the amount of absorbent material 72 fed through the absorbent material deposition station 1706 can be configured to result in absorbent cores 30 comprising absorbent material 72 disposed in amounts between 50 gsm and 1000 gsm, or between 100 gsm and 1000 gsm, or between 150 gsm and 1000 gsm, or between 200 gsm and 800 gsm, or between 250 gsm and 800 gsm, or between 300 gsm and 700 gsm, or between 350 gsm and 700 gsm, or between 400 gsm and 700 gsm, or between 450 gsm and 700 gsm, or between 500 gsm and 700 gsm, or between 400 gsm and 600 gsm, or between 500 gsm and 600 gsm, specifically reciting each 1 gsm increment within these ranges and any ranges formed therein or thereby. Such absorbent material basis weight values for absorbent cores may be particularly suitable for use in absorbent articles such as diapers and adult incontinence garments. Although, further absorbent cores that may be formed according to aspects of the present disclosure can have even smaller basis weights of absorbent material 1300, such as between 5 gsm and 50 gsm, or 5 gsm and 30 gsm, or between 10 gsm and 30 gsm.

The discharge nozzle 1800 comprises a discharge nozzle opening 1804. The discharge nozzle opening 1804 may have an opening width 1806 in the machine direction 1704. These combinations of features –the gravity feed method and the discharge nozzle opening width 1806 –may help to generate a “sheet” or “stream” 1716 of absorbent material 72 flowing toward the first substrate layer 1702. The chute opening widths 1806 may help to ensure that the stream 1716 of absorbent material 72 has a sufficient width and/or density –particularly at the points where the thermoplastic filaments 1722 contact the stream 1716, which can allow for the thermoplastic filaments 1722 to better penetrate the stream 1716 and intermix with the absorbent material 72. These configurations can help to drive beneficial properties of the resulting absorbent cores 30, as described in more detail below. In some further embodiments, air streams or air curtains may be used to help shape the stream 1716 and/or to maintain a desired width and/or density of the stream. In such embodiments, the absorbent material 72 may be directed toward the first substrate layer 1702 to some extent faster than solely by gravity, but such embodiments would still be considered to comprise a gravity feed system because the absorbent material 72 is not pneumatically or otherwise forced from the discharge nozzle opening 1804.

In one form, as the absorbent material 72 falls toward the first substrate layer 1702, thermoplastic filament applicator(s) 1718 spray thermoplastic filaments 1722 toward the falling absorbent material 72. The thermoplastic filaments 1722 intermix with the falling absorbent material 72 prior to the mixture of the absorbent material 72 and the thermoplastic filaments 1722 depositing onto the first substrate layer 1702. The method may comprise one or more than one thermoplastic filament applicator(s) 1718. A first thermoplastic filament applicator may be disposed upstream of a second thermoplastic filament applicator. FIG. 18 is a close-up schematic depiction of the absorbent material deposition station 1706. The thermoplastic filaments 1722 may generally be added to the stream of absorbent material 72 at a rate of less than 7%, or less than 6%, or less than 5%, or less than 4%, or less than 3%, or less than 2% of the weight of the absorbent material 72. In other embodiments, the thermoplastic filaments 1722 may be added to the absorbent core 30 at a level of between about 1% about and about 7%, or between about 1.5% and about 7%, or between about 2% and about 6%, or between about 2% and about 5.5%, or between about 2% and about 5%, or between about 2% and about 4.5%, or between about 2% and about 4%, or between about 2.5% and about 4%, or between about 3% and about 4% of the amount of absorbent material 72.

The thermoplastic filament applicator(s) 1718 may be configured to spray first thermoplastic filaments 1722 such that the first thermoplastic filaments 1722 contact a first side of the stream 1716 of absorbent material 72 along a portion of the stream 1716 having a length along the stream 1716. In some embodiments, the length may be insubstantial in that the first thermoplastic filaments 1722 may be sprayed as a stream which has minimal-to-no spread. However, in other embodiments, the first thermoplastic filaments 1722 may have some spread and accordingly the length along the stream 1716 could be between 2 mm and 10 mm, or between 2 mm and 6 mm, or between 2 mm and 4 mm. To allow sufficient time for the thermoplastic filaments 1722 to intermix with the stream 1716 of the absorbent material 72 prior to the mixture depositing onto the first substrate layer 1702, the superabsorbent material 1300 desirably contacts the stream 1716 at a first contact point located a distance away from the first substrate layer. The distance may be between 4 mm and 40 mm, or between 4 mm and 35 mm, or between 5 mm and 30 mm, or between 6 mm and 25 mm.

The angle at which the thermoplastic filament applicator(s) 1718 is oriented with respect to the machine direction 1704 may be important in achieving a desired level of mixing between the thermoplastic filaments 1722 and the stream 1716. Preferably, the angle is between 40 degrees and 80 degrees, or between 45 degrees and 75 degrees, or between 50 degrees and 70 degrees.

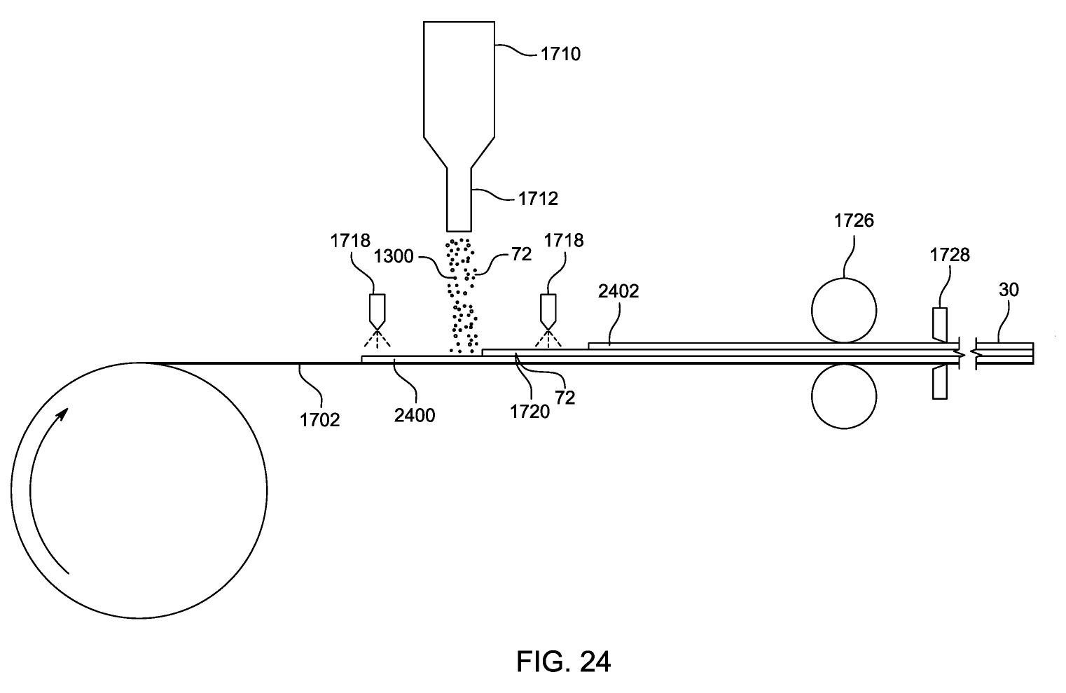

In another form, as illustrated in FIG. 24, the first thermoplastic filament applicator may be configured to direct the discharged thermoplastic filaments 1722 directly onto the first substrate layer 1702. The discharge nozzle 1800 may be configured to direct the discharged absorbent material 72, including superabsorbent material 1300 alone or in combination with other absorbent fibers, onto the first substrate layer 1702 and the discharged thermoplastic filaments 1722. In such a configuration, the thermoplastic filaments 1722 may form a first scrim layer 2400 on at least one side of the absorbent material 72. The first scrim layer may act as a structuring component, adding integrity to the otherwise loose absorbent material 72. A second thermoplastic filament applicator may be disposed after the discharge nozzle 1800 and may be configured to deposit thermoplastic filament onto the absorbent material 72 layer, forming a second scrim layer 2402 on the opposing side as the first scrim layer.

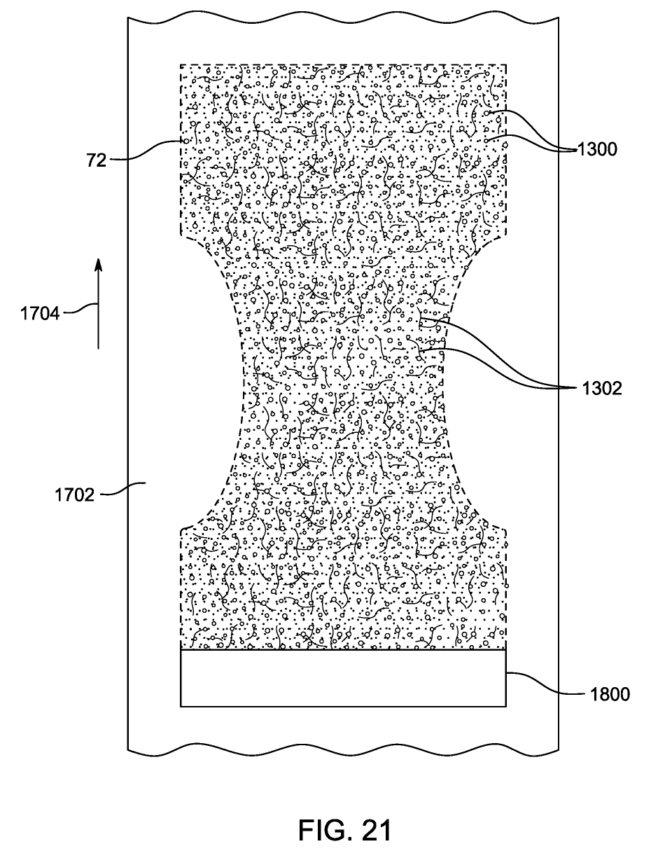

The chute 1712 may comprise one or more than one discharge nozzles 1800 that are moveable or comprise moveable components, such as applicators. The discharge nozzle(s) and/or applicators may be moveable about an axis, or where there are two or more discharge nozzles, the discharge nozzles may be moveable about one another. Referring again to FIG. 18, at least an end of the chute 1712 comprising a discharge nozzle 1800 may be configured to rotate about an axis orthogonal to the machine direction 1704. Rotation of the chute 1712 and/or discharge nozzle 1800 about an axis orthogonal to the machine direction 1704 allows for the absorbent material 72 to be deposited to create a shaped deposition area (such as, for example, trapezoidal shaped, T-shaped, I-shaped, or hourglass shaped) rather than deposited as a square or rectangular deposition area. Referring to FIGS. 19-21, the chute 1712 and/or discharge nozzle 1800 may be disposed with the opening width 1806 generally perpendicular to the machine direction 1704, such that the absorbent material 72 deposition area is generally rectangular. As the first substrate layer 1702 proceeds in the machine direction 1704, the chute 1712 and/or discharge nozzle 1800 may articulate about an axis orthogonal to the machine direction 1704, causing the area of deposition of the absorbent material 72 to narrow in the cross-machine direction, creating a shaped deposition area. Varying the speed of the first substrate layer 1702 in the machine direction 1704 and/or the speed and/or degree of swivel of the chute 1712 and/or discharge nozzle 1800 may result in various shaped deposition areas. For example, a relatively slow swivel speed may result in a gradual narrowing of the absorbent material 72 deposition area, thus forming an elongated hourglass shaped deposition area. On the other hand, a relatively fast swivel speed may result in an I-shaped deposition area or a T-shaped area if the chute 1712 and/or discharge nozzle 1800 does not return to its starting position prior to running past the end of the absorbent material 72 deposition area.

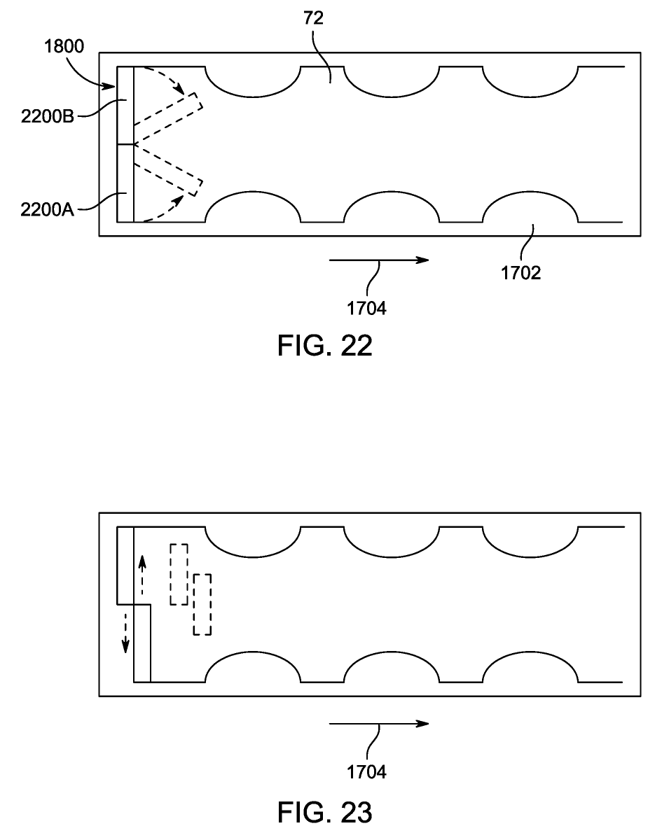

The discharge nozzle 1800 may comprise one, two, or more applicators. For example, as shown in FIG. 22, the discharge nozzle 1800 comprises two applicators – 2200A and 2200B. Each applicator 2200A and 2200B is configured to rotate about an axis perpendicular to the machine direction 1704. The two applicators 2200A and 2200B may rotate from one end proximate to the center of the first substrate layer 1702, such that a shaped deposition area may be created with edges of the deposition area narrowing from the lateral sides of the first substrate layer 1702 to create an hourglass, I-shaped, or T-shaped deposition area. Because the absorbent cores 30 are initially formed as a single sheet prior to being cut into individual absorbent cores – as shown for example in FIG. 17 – the absorbent material deposition area may define a repeated hourglass shape, a repeated T-shape, or a repeated I-shape.

In a further example, the discharge nozzle 1800 may comprise two or more applicators, wherein the two or more applicators are configured to move relative to each other in a by-pass manner – as illustrated in FIG. 23 - such that the applicators move away from the longitudinal side edges of the first substrate layer 1702 as the first substrate layer 1702 moves in the machine direction 1704. The resulting shaped deposition area may be hourglass shaped, I-shaped, or T-shaped, depending on the speed of the first substrate layer 1702 in the machine direction 1704 and/or the speed of the applicators. In addition, the by-pass manner of movement of the applicators may also allow for profiling of the absorbent material 72 in the longitudinal direction of the absorbent core as the applicators may both begin to deposit absorbent material on the same location of the core as they begin to by-pass each other. The resulting absorbent material deposition may be both shaped – hourglass, I-shaped, T-shaped, etc. – and profiled, with a greater density of absorbent material deposited in the crotch region of the absorbent core as compared to the front and back regions.

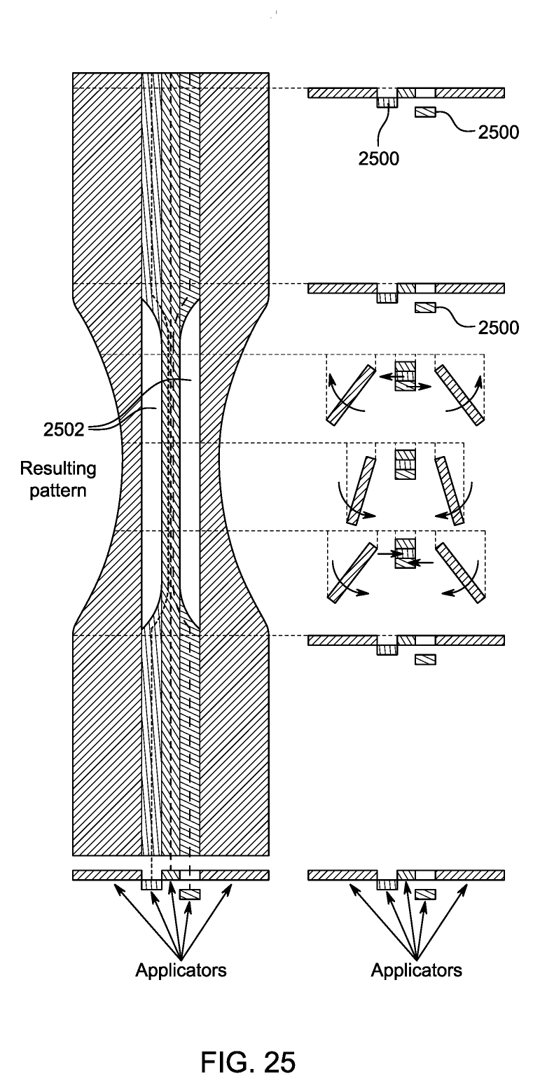

The discharge nozzle 1800 may comprise an applicator or applicators configured to form channels within the absorbent material deposition area. As illustrated in FIG. 25, for example, channel-forming applicator(s) 2500 may be disposed inboard of the absorbent material deposition area. In other words, the channel-forming applicator(s) may be surrounded on two sides by further applicators. In a form, the channel-forming applicator(s) 2500 may be configured to move relative to other adjacent, for example in a by-pass fashion as shown in FIG. 25, in order to create a substantially absorbent material-free area (channel 2502) within the absorbent material deposition area as the first substrate layer 1702 proceeds in the machine direction 1704. In another form, the channel-forming applicator(s) 2500 may be configured to stop delivering absorbent material 72 to the first substrate layer 1702 intermittently during production of absorbent cores in order to create substantially absorbent material-free channels 2500 in the absorbent material deposition area.

As the first substrate layer 1702 passes the absorbent material deposition station 1706, a deposited mixture of the thermoplastic filament 1722 and the absorbent material 72 forms as a matrix of thermoplastic filaments 1722 and absorbent material 72. Generally, the thermoplastic filaments 1722 in the mixing region form a mesh network with the absorbent material 72 captured therein. When this mesh network is deposited onto the first substrate layer, the thermoplastic filaments 1722 form a three-dimensional mesh network with the absorbent material 72 immobilized therein.

During the deposition of the mixture of absorbent material 72, vacuum energy may optionally be applied to the web first substrate layer 1702. For example, the first substrate layer 1702 may be supported by a forming surface –such as a forming belt or forming drum as is typical in the art. Vacuum energy may be applied to the forming surface such that air is drawn through the forming surface from the side where the first substrate layer 1702 is located. Accordingly, the first substrate layer 1702, along with at least a portion of the absorbent material 72 as it is falling toward the first substrate layer 1702, is drawn to the forming surface due to the applied vacuum energy. Such vacuum energy may help to control the spread of the mixture of thermoplastic filaments 1722 and absorbent material 72 as it is falling toward the first substrate layer 1702, thereby helping to form a relatively more uniform absorbent core 30. It may be preferred that the vacuum energy produces a pressure differential of greater than 0.25 m of water at the forming surface. In further embodiments, it may be more preferable for even higher pressure differentials, such as greater than 0.35 m of water, or greater than 0.5 m of water, or greater than 0.65 m of water, as measured at the forming surface.

An internal structuring web material 1400 may be applied to the deposited absorbent material 72. The internal structuring web material 1400 may generally be a lofty non-woven material and can provide structure to the absorbent core 30. In the embodiment of process 1700, only a single absorbent layer may be formed as part of the absorbent core 30. A second substrate material 1724 may be applied to the internal structuring web material 1400. A laminating adhesive 1727 may be applied to the second substrate material 1724 (or the internal structuring web material 1400 in other embodiments) by adhesive applicator 1717 prior to combining the internal structuring web material 1400 and the first substrate material 1702.

The combination of the first substrate material 1702, the deposited mixture 1720, the internal structuring web material 1400, and the second substrate material 1724 may pass through one or more nip stations 1726 to help compress the components together. In general, the nip station 1726 may apply a pressure to the combination of the first substrate material 1702, the deposited mixture 1720, the internal structuring web material 1400, and the second substrate material 1724 of between 0.5 pounds per linear inch (PLI) (88 N/m) and 1.5 PLI (263 N/m), or between 0.75 PLI (131 N/m) and 1.25 PLI (219 N/m). Such pressures may help to further connect the deposited mixture 1720 to the first and second substrate materials 1702, 1724. Although not required in all embodiments, it may be preferred for the nip station 1726 to be positioned in relatively close proximity to the material deposition station 1706 such that the adhesives retain tack when the combination of the first substrate material 1702, the deposited mixture 1720, and the second substrate material 1724 passes through the nip station 1726.

After the one or more nip stations 1726, the combination of the first substate material 1702, the deposited mixture 1720, the internal structuring web material 1400, and the second substrate material 1724 may pass to a cutting station 1728 where the connected length of the combination structure is cut into individual absorbent cores 30. These individual absorbent cores 30 may then be combined into a manufacturing process for producing the various absorbent articles described herein.

The absorbent core formation process 1700 may employ two absorbent material deposition stations 1706, 1730. The resulting absorbent core 30 may then comprise a first deposited mixture 1720 and a second deposited mixture 1732 that may be separated by an internal structuring web material 1400. The total absorbent material 72 may be divided evenly between the first deposited mixture 1720 and the second deposited mixture 1732, or one layer may contain more absorbent material 72 than the other. The second absorbent material deposition station 1730 may be configured the same as the first absorbent material deposition station 1706, as discussed above.

The second substrate material 1724 may be coupled to the internal structuring web material 1400 or the deposited mixture 1720, 1732 of the absorbent material 72 and the thermoplastic filaments 1722 to form the absorbent core 30. Some alternative embodiments according to aspects of the present disclosure may forgo the second substrate material 1724 altogether. In such embodiments, the first substrate material 1702 may be wide enough that, after the mixture 1720 is deposited onto the first substrate material 1702, the first substrate material 1702 is wrapped around the deposited mixture 1720 to form the absorbent core 30.

In at least some embodiments, the absorbent material 72 is able to be stabilized within the three-dimensional mesh network of the thermoplastic filaments 1722 to a sufficient degree that additional adhesives may not be needed. Such embodiments generally allow for faster intake times relative to more traditional absorbent structures. For example, less adhesive overall may be utilized in the absorbent cores 30 of the present disclosure. Adhesives are generally hydrophobic and block fluid flow into absorbent cores. Accordingly, a lesser amount of adhesive may help to improve fluid uptake times.

### Packages

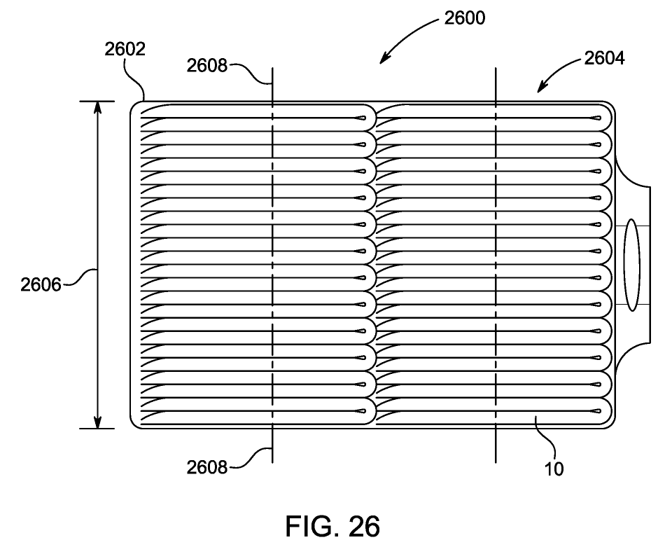

Referring to FIG. 26, the absorbent articles 10 of the present disclosure may be placed into packages 2600. The packages 2600 may comprise polymeric films, paper, and/or other materials 2602. Graphics and/or indicia relating to properties of the absorbent articles 10 may be formed on, printed on, positioned on, and/or placed on outer portions of the packages 2600. Each package 2600 may comprise a plurality of absorbent articles 10. The absorbent articles 10 may be packed under compression so as to reduce the size of the packages, while still providing an adequate number of absorbent articles per package. By packaging the absorbent articles under compression, caregivers can easily handle and store the packages, while also providing distribution savings to manufacturers owing to the size of the packages. The packages may comprise polymeric films comprising recycled material, such as about 20% to about 100%, about 30% to about 90%, about 30% to about 80%, about 40% to about 60%, or about 50% recycled material. The recycled material may comprise post-industrial recycled material (PIR) and/or post-consumer recycled material (PCR). In some instances, the polymeric films used for the packages may comprise two outer layers and one or more inner layers. The one or more inner layers may comprise the recycled material or may comprise more recycled material than the outer layers. The recycled material may comprise recycled polyethylene. The recycled material may comprise recycled polyethylene PIR from trim from the packaging operation.

The package material may comprise paper, paper based material, paper with one or more barrier layers, or a paper/film laminate. The package material may be in the range of about 50 gsm to about 100 gsm or about 70 gsm to about 90 gsm and the one or more barrier layers may be in the range of about 3 gsm to about 15 gsm. The paper based package material with or without one or more barrier layers may exhibit a machine direction tensile strength of at least 5.0 kN/m, a machine direction stretch of at least 3 percent, a cross-machine direction tensile strength of at least 3 kN/m, and a cross-direction stretch at break of at least 4 percent, each as determined via ISO 1924-3.

The paper based package material or paper based package material comprising a barrier layer or film may be recyclable or recyclable in normal paper recycling operations. The recyclability extent of the paper based package may be determined via recyclable percentage. The paper based package of the present disclosure may exhibit recyclable percentages of 70 percent or greater, 80 percent or greater, or 90 percent or greater. The paper based package of the present disclosure may have a recyclable percentage of between 70 percent to about 99.9 percent, between about 80 percent to about 99.9 percent, or between about 90 percent to about 99.9 percent. In one example, the package material of the present disclosure may exhibit a recyclable percentage of from about 95 percent to about 99.9 percent, from about 97 percent to about 99.9 percent, or from about 98 percent to about 99.9 percent. The recyclable percentage of the paper based package may be determined via test PTS-RH:021/97 (Draft Oct. 2019) under category II, as performed by Papiertechnische Stiftung located at Pirnaer Strasse 37, 01809 Heidenau, Germany. In another instance, the paper based packages of the present disclosure may exhibit an overall “pass” test outcome as determined by PTS-RH:021/97 (Draft Oct. 2019) under category II method. Any of the paper based packages may have opening features, such as lines of perforation, and may also have handles.

The absorbent articles 10 of the present disclosure may be disposed within packages 2600 and/or packaging material 2602 to form a stack 2604 or a plurality of stacks of absorbent articles 10, as shown in FIG. 26. A stack 2604 of absorbent articles 10 of the present disclosure may exhibit an In-Bag Stack Height of from about 70 mm to about 200 mm, or from about 72 mm to about 185 mm, or from about 74 mm to about 165 mm, or from about 76 mm to about 150 mm, specifically reciting all values within these ranges and any ranges formed therein or thereby. The In-Bag Stack Height = (the width 2606 of the package 2600 / pad count per stack) x 10. The package width 2606 is defined as the maximum distance between the two highest bulging points along the same compression stack axis 2608 of an absorbent article package 2600.

### Arrays

“Array” means a display of packages comprising disposable absorbent articles of different article constructions (e.g., different elastomeric materials [compositionally and/or structurally] in the side panels, back ears, side flaps and/or belts flaps, different graphic elements, different product structures, fasteners, waistbands, or lack thereof). The packages may have the same brand and/or sub-brand and/or the same trademark registration and/or having been manufactured by or for a common manufacturer and the packages may be available at a common point of sale (e.g. oriented in proximity to each other in a given area of a retail store). An array is marketed as a line-up of products normally having like packaging elements (e.g., packaging material type, film, paper, dominant color, design theme, etc.) that convey to consumers that the different individual packages are part of a larger line-up. Arrays often have the same brand, for example, “Huggies,” and same sub-brand, for example, “Pull-Ups.” A different product in the array may have the same brand “Huggies” and the sub-brand “Little Movers.” The differences between the “Pull-Ups” product of the array and the “Little Movers” product in the array may include product form, application style, different fastening designs or other structural elements intended to address the differences in physiological or psychological development. Furthermore, the packaging is distinctly different in that “Pull-Ups” is packaged in a predominately blue or pink film bag and “Little Movers” is packaged in a predominately red film bag.

Further regarding “Arrays,” as another example an array may be formed by different products having different product forms manufactured by the same manufacturer, for example, “Kimberly-Clark”, and bearing a common trademark registration for example, one product may have the brand name “Huggies,” and sub-brand, for example, “Pull-Ups.” A different product in the array may have a brand/sub-brand “Good Nites” and both are registered trademarks of The Kimberly-Clark Corporation and/or are manufactured by Kimberly-Clark. Arrays also often have the same trademarks, including trademarks of the brand, sub-brand, and/or features and/or benefits across the line-up. “On-line Array” means an “Array” distributed by a common on-line source.

### Sanitary Napkin

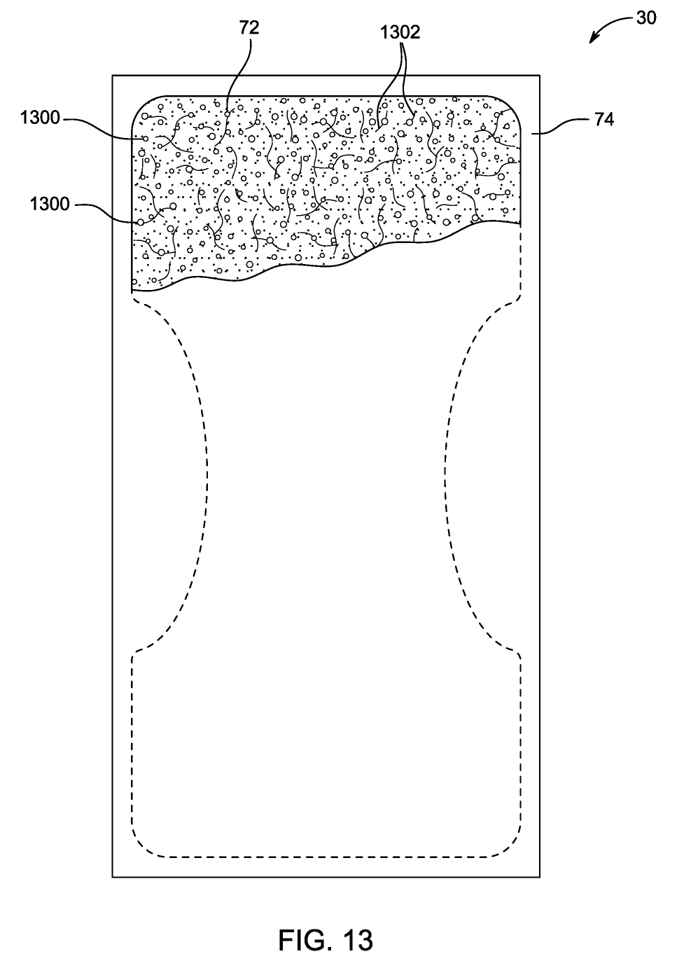

Referring to FIG. 12, an absorbent article of the present disclosure may be a sanitary napkin 110. The sanitary napkin 110 may comprise a liquid permeable topsheet 114, a liquid impermeable, or substantially liquid impermeable, backsheet 116, and an absorbent core 118. The liquid impermeable backsheet 116 may or may not be vapor permeable. The absorbent core 118 may have any or all of the features described herein with respect to the absorbent core 30 and, in some forms, may have a secondary topsheet 119 (STS) instead of the acquisition materials disclosed above. The STS 119 may comprise one or more channels, as described above (including the embossed version). In some forms, channels in the STS 119 may be aligned with channels in the absorbent core 118. The sanitary napkin 110 may also comprise wings 120 extending outwardly with respect to a longitudinal axis 180 of the sanitary napkin 110. The sanitary napkin 110 may also comprise a lateral axis 190. The wings 120 may be joined to the topsheet 114, the backsheet 116, and/or the absorbent core 118. The sanitary napkin 110 may also comprise a front edge 122, a back edge 124 longitudinally opposing the front edge 122, a first side edge 126, and a second side edge 128 longitudinally opposing the first side edge 126. The longitudinal axis 180 may extend from a midpoint of the front edge 122 to a midpoint of the back edge 124. The lateral axis 190 may extend from a midpoint of the first side edge 128 to a midpoint of the second side edge 128. The sanitary napkin 110 may also be provided with additional features commonly found in sanitary napkins as is known in the art.

### Fasteners

Referring to FIG. 1, the back ears 42 may comprise fasteners 46. The fasteners 46 may be configured to cooperate with the landing zone area or landing zone material 44 on the garment-facing surface of the front waist region 12 of the absorbent article 10. Additionally, the absorbent article may comprise one or more secondary fasteners. The secondary fasteners may be disposed on the outer cover material 40 or a component of the absorbent article, such as the landing zone 44. The secondary fasteners may be a separate material joined to the absorbent article. The secondary fasteners may be integrally formed from a material of the absorbent article. For example, the secondary fasteners may comprise one or more hooks or protrusions formed from the material of the outer cover material 40, the landing zone material 44, or a film. The hooks or protrusions may be formed using the process described in U.S. Pat. No. 8,784,722 to Rocha et al. The secondary fasteners may be configured to cooperate with a portion of the back ears. For example, a secondary fastener comprising hooks or protrusions are configured to engage the nonwoven material of the back ear. It is to be appreciated that the secondary fasteners may be any mechanical fastener that is configured to cooperate with a component of the absorbent article.

### Bio-Based Content for Components

Components of the absorbent articles described herein may at least partially be comprised of bio-based content as described in U.S. Pat. Appl. No. 2007/0219521A1. For example, the superabsorbent polymer component may be bio-based via their derivation from bio-based acrylic acid. Bio-based acrylic acid and methods of production are further described in U.S. Pat. Appl. Pub. No. 2007/0219521 and U.S. Pat. Nos. 8,703,450; 9,630,901 and 9,822,197. Other components, for example nonwoven and film components, may comprise bio-based polyolefin materials. Bio-based polyolefins are further discussed in U.S. Pat. Appl. Pub. Nos. 2011/0139657, 2011/0139658, 2011/0152812, and U.S. Pat. Nos. 10,166,312 and 9,169,366. Example bio-based polyolefins for use in the present disclosure comprise polymers available under the designations SHA7260^{TM}, SHE150^{TM}, or SGM9450F^{TM} (all available from Braskem S.A.).

An absorbent article component may comprise a bio-based content value from about 10% to about 100%, from about 25% to about 100%, from about 40% to about 100%, from about 50% to about 100%, from about 75% to about 100%, or from about 90% to about 100%, for example, using ASTM D6866-10, method B.

### Recycle Friendly and Bio-Based Absorbent Articles

Components of the absorbent articles described herein may be recycled for other uses, whether they are formed, at least in part, from recyclable materials. Examples of absorbent article materials that may be recycled are nonwovens, films, fluff pulp, and superabsorbent polymers. The recycling process may use an autoclave for sterilizing the absorbent articles, after which the absorbent articles may be shredded and separated into different byproduct streams. Example byproduct streams may comprise plastic, superabsorbent polymer, and cellulose fiber, such as pulp. These byproduct streams may be used in the production of fertilizers, plastic articles of manufacture, paper products, viscose, construction materials, absorbent pads for pets or on hospital beds, and/or for other uses. Further details regarding absorbent articles that aid in recycling, designs of recycle friendly diapers, and designs of recycle friendly and bio-based component diapers, are disclosed in U.S. Pat. No. 11,433,158, issued on September 6, 2022.

### Combinations

1. A method of forming an absorbent core, the method comprising:

advancing a first substrate layer in a machine direction, the first substrate layer forming a first side of the absorbent core;

feeding a first stream of absorbent material comprising superabsorbent particles and thermoplastic filaments toward the first substrate layer;

depositing the first stream of absorbent material onto the first substrate layer through at least one discharge nozzle, wherein the at least one discharge nozzle is configured to rotate about an axis orthogonal to the machine direction, and wherein the deposited first stream of absorbent material forms a first absorbent material layer;

applying a second substrate layer to form a second side of the absorbent core opposite the first side of the absorbent core.

2. The method of paragraph 1, wherein the absorbent material is air-mixed prior to deposition onto the first substrate layer.

3. The method of paragraph 1, wherein the at least one discharge nozzle configured to rotate about an axis orthogonal to the machine direction is designed to deposit the absorbent material at varying widths on the first material layer along the machine direction.

4. The method of paragraphs 1-3, wherein the step of depositing the absorbent material onto the first substrate layer through at least one discharge nozzle creates an absorbent material deposition area defining a deposition area perimeter, and wherein at least a portion of the deposition area perimeter is curvilinear.

5. The method of paragraph 4, wherein at least a portion of a first longitudinal side and at least a portion of a second longitudinal side of the deposition area perimeter are curvilinear.

6. The method of paragraph 4, wherein at least a portion of the absorbent material deposition area defines an hourglass shape, a T-shape, or an I-shape.

7. The method of any one of the preceding paragraphs, wherein the absorbent material further comprises absorbent pulp fibers and/or staple fibers.

8. The method of any one of the preceding paragraphs, wherein the first substrate layer is advanced on a forming surface, and wherein a pressure differential is created at the forming surface.

9. The method of paragraph 8, wherein the forming surface is a forming belt or a forming drum.

10. The method of paragraphs 8 or 9, wherein the pressure differential is created by application of vacuum energy.

11. The method of paragraphs 8, 9, or 10, wherein the pressure differential is greater than 0.25 m of water, greater than 0.35 m of water, greater than 0.5 m of water, or greater than 0.65 m of water, as measured at the forming surface.

12. The method of any of the preceding paragraphs, wherein the second substrate layer is applied in face-to-face relationship with the first absorbent material layer.

13. The method of any one of paragraphs 1-12, further comprising the steps of:

applying an internal structuring web material in a face-to-face relationship with the first absorbent layer;

depositing a second stream of absorbent material onto the internal structuring web material through at least one discharge nozzle, wherein the at least one discharge nozzle is configured to rotate about an axis orthogonal to the machine direction, and wherein the deposited second stream of absorbent material forms a second absorbent material layer; and

applying the second substrate layer in face-to-face relationship with the second absorbent material layer.

14. The method of claim 13, wherein the internal structuring web material is a nonwoven material.

15. The method of any one of the preceding paragraphs, wherein the first absorbent material layer and/or the second absorbent material layer comprises at least one channel substantially free of absorbent material.

16. A method of forming an absorbent core, the method comprising:

advancing a first substrate layer in a machine direction, the first substrate layer forming a first side of the absorbent core;

feeding a first stream of absorbent material comprising superabsorbent particles and thermoplastic filaments toward the first substrate layer;

depositing the first stream of absorbent material onto the first substrate layer through at least one discharge nozzle, wherein the at least one discharge nozzle comprises an applicator or a plurality of applicators, wherein the applicator or the plurality of applicators move about an axis orthogonal to the machine direction, and wherein the deposited first stream of absorbent material forms a first absorbent material layer;

applying a second substrate layer to form a second side of the absorbent core opposite the first side of the absorbent core.

17. The method of paragraph 16, wherein the discharge nozzle comprises two applicators, and wherein each applicator rotates from one end proximate the center of the first substrate layer to form a shaped absorbent material deposition area.

18. The method of paragraph 17, wherein the absorbent material deposition area at least partially forms an hourglass shape, an I-shape, or a T-shape.

19. The method of paragraph 17, wherein the absorbent material deposition area forms a repeated hourglass shape, a repeated I-shape, or a repeated T-shape.

20. The method of any of paragraphs 16-19, wherein the second substrate layer is applied in a face-to-face relationship with the first absorbent material layer.

21. A method of forming an absorbent core, the method comprising:

advancing a first substrate layer in a machine direction, the first substrate layer forming a first side of the absorbent core;

feeding a first stream of absorbent material comprising superabsorbent particles and thermoplastic filaments toward the first substrate layer;

depositing the first stream of absorbent material onto the first substrate layer through at least one discharge nozzle, wherein the at least one discharge nozzle comprises a plurality of applicators, wherein the plurality of applicators move relative to each other in a by-pass manner, and wherein the deposited first stream of absorbent material forms a first absorbent material layer;

applying a second substrate layer to form a second side of the absorbent core opposite the first side of the absorbent core.

22. The method of paragraph 21, wherein the absorbent material deposition area at least partially forms an hourglass shape, an I-shape, or a T-shape.

23. The method of paragraph 21, wherein the absorbent material deposition area forms a repeated hourglass shape, a repeated I-shape, or a repeated T-shape.

24. The method of any one of paragraphs 21-23, further comprising the steps of:

applying an internal structuring web material in a face-to-face relationship with the first absorbent layer;

depositing a second stream of absorbent material onto the internal structuring web material through at least one discharge nozzle, wherein the deposited second stream of absorbent material forms a second absorbent material layer; and

applying the second substrate layer in face-to-face relationship with the second absorbent material layer.

25. The method of paragraph 24, wherein the internal structuring web material is a nonwoven material.

26. The method of paragraph 24, wherein the first absorbent material layer and/or the second absorbent material layer comprises at least one channel substantially free of absorbent material.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of this present disclosure.

What is claimed is:

1. A method of forming an absorbent core, the method comprising:
advancing a first substrate layer in a machine direction, the first substrate layer forming a first side of the absorbent core;
feeding a first stream of absorbent material comprising superabsorbent particles and thermoplastic filaments toward the first substrate layer;
depositing the first stream of absorbent material onto the first substrate layer through at least one discharge nozzle, wherein the at least one discharge nozzle is configured to rotate about an axis orthogonal to the machine direction, and wherein the deposited first stream of absorbent material forms a first absorbent material layer;
applying a second substrate layer to form a second side of the absorbent core opposite the first side of the absorbent core.

2. The method of claim 1, wherein the absorbent material is air-mixed prior to deposition onto the first substrate layer.

3. The method of claim 1, wherein the at least one discharge nozzle configured to rotate about an axis orthogonal to the machine direction is designed to deposit the absorbent material at varying widths on the first material layer along the machine direction.

4. The method of claim 1, wherein the step of depositing the absorbent material onto the first substrate layer through at least one discharge nozzle creates an absorbent material deposition area defining a deposition area perimeter, and wherein at least a portion of the deposition area perimeter is curvilinear.

5. The method of claim 4, wherein at least a portion of a first longitudinal side and at least a portion of a second longitudinal side of the deposition area perimeter are curvilinear.

6. The method of claim 4, wherein at least a portion of the absorbent material deposition area defines an hourglass shape, a T-shape, or an I-shape.

7. The method of claim 1, wherein the absorbent material further comprises absorbent pulp fibers and/or staple fibers.

8. The method of claim 1, wherein the first substrate layer is advanced on a forming surface, and wherein a pressure differential is created at the forming surface.

9. The method of claim 8, wherein the forming surface is a forming belt or a forming drum.

10. The method of claim 8, wherein the pressure differential is created by application of vacuum energy.

11. The method of claim 8, wherein the pressure differential is greater than 0.25 m of water, as measured at the forming surface.

12. The method of claim 1, wherein the second substrate layer is applied in face-to-face relationship with the first absorbent material layer.

13. The method of claim 1, further comprising the steps of:
applying an internal structuring web material in a face-to-face relationship with the first absorbent layer;
depositing a second stream of absorbent material onto the internal structuring web material through at least one discharge nozzle, wherein the at least one discharge nozzle is configured to rotate about an axis orthogonal to the machine direction, and wherein the deposited second stream of absorbent material forms a second absorbent material layer; and
applying the second substrate layer in face-to-face relationship with the second absorbent material layer.

14. The method of claim 1, wherein the first absorbent material layer comprises at least one channel substantially free of absorbent material.

15. A method of forming an absorbent core, the method comprising:
advancing a first substrate layer in a machine direction, the first substrate layer forming a first side of the absorbent core;
feeding a first stream of absorbent material comprising superabsorbent particles and thermoplastic filaments toward the first substrate layer;
depositing the first stream of absorbent material onto the first substrate layer through at least one discharge nozzle, wherein the at least one discharge nozzle comprises an applicator or a plurality of applicators, wherein the applicator or the plurality of applicators move about an axis orthogonal to the machine direction, and wherein the deposited first stream of absorbent material forms a first absorbent material layer;
applying a second substrate layer to form a second side of the absorbent core opposite the first side of the absorbent core.

16. The method of claim 15, wherein the discharge nozzle comprises two applicators, and wherein each applicator rotates from one end proximate the center of the first substrate layer to form a shaped absorbent material deposition area.

17. The method of claim 16, wherein the absorbent material deposition area at least partially forms an hourglass shape, an I-shape, or a T-shape.

18. The method of claim 16, wherein the absorbent material deposition area forms a repeated hourglass shape, a repeated I-shape, or a repeated T-shape.

19. The method of claim 15, wherein the second substrate layer is applied in a face-to-face relationship with the first absorbent material layer.

20. A method of forming an absorbent core, the method comprising:
advancing a first substrate layer in a machine direction, the first substrate layer forming a first side of the absorbent core;
feeding a first stream of absorbent material comprising superabsorbent particles and thermoplastic filaments toward the first substrate layer;
depositing the first stream of absorbent material onto the first substrate layer through at least one discharge nozzle, wherein the at least one discharge nozzle comprises a plurality of applicators, wherein the plurality of applicators move relative to each other in a by-pass manner, and wherein the deposited first stream of absorbent material forms a first absorbent material layer;
applying a second substrate layer to form a second side of the absorbent core opposite the first side of the absorbent core.
